(19) United States
(12) Patent Application Publication
(10) Pub. No.: US 2015/0150892 A1
(43) Pub. Date: Jun. 4, 2015

(54) METHODS AND PRODUCTS RELATED TO LUNG CANCER

(71) Applicant: Massachusetts Institute of Technology, Cambridge, MA (US)

(72) Inventors: Tyler E. Jacks, Newton, MA (US); Wen Xue, Watertown, MA (US); Etienne Meylan, Ecublens (CH); Trudy Gale Oliver, Salt Lake City, UT (US); David Feldser, Somerville, MA (US); Monte Winslow, Palo Alto, CA (US)

(73) Assignee: Massachusetts Institute of Technology, Cambridge, MA (US)

(21) Appl. No.: 14/405,985

(22) PCT Filed: Jun. 7, 2013
(86) PCT No.: PCT/US2013/044833

§ 371 Date: Dec. 5, 2014

Related U.S. Application Data
(60) Provisional application No. 61/656,714, filed on Jun. 7, 2012.

(51) Int. Cl. A61K 31/69; A61K 45/06; G01N 33/574; C12N 15/113; C12Q 1/68; A61K 31/277; A61K 31/713

(52) U.S. Cl. CPC A61K 31/69; A61K 31/277; A61K 45/06; A61K 31/713; C12N 15/113; C12Q 1/6886; G01N 33/57423; C12N 2310/531; C12Q 2600/158; G01N 2333/91045; G01N 2333/521; G01N 2333/70542; G01N 2333/96433; G01N 2333/8121; G01N 2333/91194

(57) ABSTRACT

The invention relates to methods and related products for treatment and determining modes of treatment for cancer. Preferably the methods relate to the inhibition of the NF-κB pathway in lung cancer.

### RELATED APPLICATIONS

This application claims priority under 35 U.S.C. §119 to U.S. provisional patent application U.S. Ser. No. 61/656,714, filed Jun. 7, 2012, entitled “METHODS AND PRODUCTS RELATED TO LUNG CANCER” the entire contents of which is incorporated herein by reference.

### BACKGROUND OF INVENTION

Lung cancer is the leading cause of cancer death worldwide, non-small-cell lung cancer (NSCLC) representing 85% of lung cancer cases. Lung adenocarcinoma, a histologic class of NSCLC, is associated with recurrent mutations in several well-defined oncogenes and tumor suppressor genes. Oncogenic Kras mutations occur in 25% of lung adenocarcinomas, and inactivating mutations in the tumor suppressor gene p53 (TP53) are found in 50% of cases (1).

The 5-year survival rate of individuals diagnosed with lung cancer in the United States is poor, at only ˜15%, and the prognosis is even worse for individuals who have a diagnosis of advanced disease (2). Although much effort has been devoted to developing targeted therapies for lung cancer, few have proven effective thus far (3). Recent successful targeted therapies include the epidermal growth factor receptor (EGFR) inhibitor gefitinib/erlotinib for patients with EGFR mutation (4) and anaplastic lymphoma kinase (ALK) inhibitors for patients with EML4-ALK translocations (5). Yet, to date, no targeted therapies have been used effectively against Kras mutant lung cancer.

### SUMMARY OF INVENTION

The invention, in some aspects is a method for selecting a course of treatment of a subject having cancer. In some aspects the invention is a method involving determining a level of NF-κB in a subject having lung cancer, and administering to the subject an effective amount of an NF-κB inhibitor to treat the subject if the subject has a higher than normal level of NF-κB. The NF-κB inhibitor in some embodiments is bortezomib. In other embodiments the NF-κB inhibitor is Bay-117082.

In some embodiments the method involves detecting a level of a marker gene expressed in a sample of cancerous tissue from the subject. The marker gene may be, for instance, HMG-CoA synthase 2, Cxcl15, 5330417C22Rik, Cp, Vcam1, Pamr1, C1s, Tnip3, Serpinb98, and/or Sult1a1. In some embodiments when the level of the marker gene is at least twice a baseline level, the subject is administered a chemotherapeutic agent.

In other aspects the invention is a method involving detecting a level of a marker gene expressed in a sample of cancerous tissue from a subject having lung cancer, wherein if the level of the marker gene is at least twice a baseline level the cancerous tissue is resistant to treatment with a NF-κB inhibitor, wherein the marker gene is a gene selected from the list presented in Table 1, and developing a therapeutic strategy for the subject based on the level of the marker gene. In some embodiments the marker gene is HMG-CoA synthase 2, Cxcl15, 5330417C22Rik, Cp, Vcam1, Pamr1, C1s, Tnip3, Serpinb98, and/or Sult1a1. In some embodiments the method involves administering to the subject a chemotherapeutic agent.

A method involving administering to a subject having lung cancer an NF-κB inhibitor and an HMG-CoA synthase 2 inhibitor in an effective amount to treat the cancer is provided in other aspects of the invention. In some embodiments the HMG-CoA synthase 2 inhibitor is an siRNA to HMG-CoA synthase 2. In other embodiments the HMG-CoA synthase 2 inhibitor is an shRNA to HMG-CoA synthase 2.

A method involving administering to a subject having lung cancer an NF-κB inhibitor and a NF-κB responsive agent in an effective amount to treat the cancer is provided in other aspects of the invention. In some embodiments the NF-κB responsive agent is an siRNA to a gene in Table 1. In other embodiments the NF-κB responsive agent is an shRNA to a gene in Table 1.

In some embodiments the NF-κB responsive gent is an inhibitor of a compound selected from the group consisting of Cxcl15, Hmgcs2, 5330417C22Rik, Cp, Vcam1, Pamr1, C1s, Tnip3, Serpinb9b, Sult1a1, Bicc1, Gbp2, and Msrb3.

In some embodiments the NF-κB responsive gent is an inhibitor of a compound selected from the group consisting of Cxcl15, Hmgcs2, 5330417C22Rik, Cp, Vcam1, Pamr1, C1s, Tnip3, Serpinb9b, Sult1a1, Bicc1, Gbp2, Msrb3, D0H4S114, Serpinb9, Cyp4a12b, LOC100045065, C3, Glipr2, Ptgs1, Trp53inp1, Gm5077, Ppp2r2b, Tgm2, Spon2, Dmbt1, Ablim3, and Klhl24.

In some embodiments the NF-κB responsive gent is an inhibitor of a compound selected from the group consisting of Cxcl15, Hmgcs2, 5330417C22Rik, Cp, Vcam1, Pamr1, C1s, Tnip3, Serpinb9b, Sult1a1, Bicc1, Gbp2, Msrb3, D0H4S114, Serpinb9, Cyp4a12b, LOC100045065, C3, Glipr2, Ptgs1, Trp53inp1, Gm5077, Ppp2r2b, Tgm2, Spon2, Dmbt1, Ablim3, Klhl24, Lbp, Atp2b4, Serpinb6b, Trp53inp2, Sepp1, Pcdhb16, Chst15, Thbs1, Pcdhb19, Ype12, Masp1, Heg1, Cacna1g, Tcn2, Cyp4b1, Cdon, Synpo, Mras, Gpd1, Sesn3, Gpr146, Hexa, A330074K22Rik, Gstt1, Zfp704, Slc7a2, and Stra6.

In some embodiments the NF-κB responsive gent is an inhibitor of a compound selected from the group consisting of Cxcl15, Hmgcs2, 5330417C22Rik, Cp, Vcam1, Pamr1, C1s, Tnip3, Serpinb9b, Sult1a1, Bicc1, Gbp2, Msrb3, D0H4S114, Serpinb9, Cyp4a12b, LOC100045065, C3, Glipr2, Ptgs1, Trp53inp1, Gm5077, Ppp2r2b, Tgm2, Spon2, Dmbt1, Ablim3, Klhl24, Lbp, Atp2b4, Serpinb6b, Trp53inp2, Sepp1, Pcdhb16, Chst15, Thbs1, Pcdhb19, Ype12, Masp1, Heg1, Cacna1g, Tcn2, Cyp4b1, Cdon, Synpo, Mras, Gpd1, Sesn3, Gpr146, Hexa, A330074K22Rik, Gstt1, Zfp704, Slc7a2, Stra6, Gda, Sned1, Gpr126, Pcdhb18, Fmo1, Ccl2, Npr2, A930001N09Rik, Sat1, Plscr2, Antxr1, Pcdhb20, Fam20c, Gstm7, Tmie, Ecscr, Tcp11l2, Man1c1, Tnfsf13, Ppargc1a, Rorc, Csrp1, Tgfb2, Clec2d, Zfp3611, Arid5b, Slc40a1, Akr1c18, Creg1, Prg4, I118, Irgm1, C1rb, Srgap1, Aldh6a1, Tifa, Gas6, Glipr1, Fam107a, Pik3ip1, 4933426M11Rik, Dusp16, Sorbs2, Calcoco1, Trf, Xaf1, Gstm2, Cyp4a12a, C1r, Lamb2, Pgcp, Gm4055, Sox4, Wnt4, Ddx58, and Gm14443.

In other embodiments the NF-κB responsive gent is an inhibitor of a compound selected from the group consisting of Cxcl15, Hmgcs2, 5330417C22Rik, Cp, Vcam1, Pamr1, C1s, Tnip3, Serpinb9b, Sult1a1, Bicc1, Gbp2, Msrb3, D0H4S114, Serpinb9, Cyp4a12b, LOC100045065, C3, Glipr2, Ptgs1, Trp53inp1, Gm5077, Ppp2r2b, Tgm2, Spon2, Dmbt1, Ablim3, Klhl24, Lbp, Atp2b4, Serpinb6b, Trp53inp2, Sepp1, Pcdhb16, Chst15, Thbs1, Pcdhb19, Ype12, Masp1, Heg1, Cacna1g, Tcn2, Cyp4b1, Cdon, Synpo, Mras, Gpd1, Sesn3, Gpr146, Hexa, A330074K22Rik, Gstt1, Zfp704, Slc7a2, Stra6, Gda, Sned1, Gpr126, Pcdhb18, Fmo1, Ccl2, Npr2, A930001N09Rik, Sat1, Plscr2, Antxr1, Pcdhb20, Fam20c, Gstm7, Tmie, Ecscr, Tcp11l2, Man1c1, Tnfsf13, Ppargc1a, Rorc, Csrp1, Tgfb2, Clec2d, Zfp3611, Arid5b, S1c40a1, Akr1c18, Creg1, Prg4, I118, Irgm1, C1rb, Srgap1, Aldh6a1, Tifa, Gas6, Glipr1, Fam107a, Pik3ip1, 4933426M11Rik, Dusp16, Sorbs2, Calcoco1, Trf, Xaf1, Gstm2, Cyp4a12a, C1r, Lamb2, Pgcp, Gm4055, Sox4, Wnt4, Ddx58, Gm14443, Crot, Sort1, Ece1, Edn1, Dusp1, Rasl11a, Parp8, 5133401N09Rik, Bmf, Pion, 9330159M07Rik, Vnn1, Serinc5, Chsy3, Ppfibp2, Tnfrsf9, 2010011120Rik, Jun, Acot2, Faah, Phyh, Arhgap26, Ype15, Cald1, BC023202, Mxd4, Thra, Nedd9, Tmem140, Il6st, Ctns, Epas1, Timp3, Zfyve1, Itih2, Ephb3, D4Bwg0951e, Igfbp7, Pik3r3, Ccpg1, Akap13, Wfdcl, Bmp4, Abca8b, Prss23, Thrb, Pkd2, LOC381508, Txnip, Tapbp, Fbxo32, Apbb3, Zcwpw1, Pik3r1, Pdzk1ip1, Fosl2, Epdr1, Vwa5a, Gm3932, 2410066E13Rik, Atxn1, Ntn4, Galc, Abcg2, Ptpn14, Mcc, Clec2g, Bco2, Parp3, Crebl2, Fam168a, Ifi2711, Pts, Atp1b1, Lmbrd1, 1700112E06Rik, Zbtb10, Pygb, Dpp7, H19, Lamp2, Sesn1, Malat1, 4833442J19Rik, Adamts9, Irs1, Slc31a2, 9230110C19Rik, Aplp2, Ggh, Cerk, Parp12, Rhou, Zhx1, 1110028C15Rik, Hexb, Foxo3, Fbx120, Idua, Gabarap11, Zfp503, Ahnak2, Clqtnf6, Cd1d1, Nfkbia, Arsk, Prtg, B930095G15Rik, Vasn, 0610040J01Rik, Dynll2, Ptprk, Slc16a4, AW549877, Zfp579, 1810020D17Rik, Slc35f5, Igtp, Rnf144b, Slc16a10, D14Ertd436e, Gm6813, Gm14057, 9230105E10Rik, BC031353, Slc25a23, Rab2b, 2210408F21Rik, Ncoa1, B4galt1, Tgfbr2, Ctso, Gbp1, Ypel1, Stx16, Acox3, Tmem57, and Ahr.

In some embodiments the subject has lung cancer with mutations in Kras and/or p53.

In some embodiments, the marker genes comprise at least one, at least two, at least three, at least four, at least five, at least six, at least seven, or at least eight genes selected from Table 1.

According to some aspects of the invention, arrays are provided that comprise, or consist essentially of, oligonucleotide probes that hybridize to nucleic acids having sequence correspondence to mRNAs of at least two, at least three, at least four, at least five, at least six, at least seven, at least eight, at least nine, at least ten, at least twenty, at least 30, at least 40, at least 50, at least 60, at least 70, at least 80, or at least 90 genes selected from the marker genes selected from Table 1.

According to some aspects of the invention, arrays are provided that comprise, or consist essentially of, antibodies that bind specifically to proteins encoded by at least two, at least three, at least four, at least five, at least six, at least seven, at least eight, at least nine, at least ten, at least twenty, at least 30, at least 40, at least 50, at least 60, at least 70, at least 80, or at least 90 genes selected from the marker genes selected from Table 1.

According to some aspects of the invention, methods are provided for monitoring progression of a cancer or of monitoring the effectiveness of a cancer therapy in an individual in need thereof. In some embodiments, the methods involve (a) obtaining a clinical sample from the individual; (b) determining expression levels of one or of a plurality of marker genes in the clinical sample using an expression level determining system, (c) comparing each expression level determined in (b) with an appropriate reference level, in which the results of the comparison are indicative of the extent of progression of the cancer in the individual or of the effectiveness of a cancer therapy in the individual.

In some embodiments the marker gene is selected from the group consisting of Cxcl15, Hmgcs2, 5330417C22Rik, Cp, Vcam1, Pamr1, C1s, Tnip3, Serpinb9b, Sult1a1, Bicc1, Gbp2, and Msrb3.

In some embodiments the marker gene is selected from the group consisting of Cxcl15, Hmgcs2, 5330417C22Rik, Cp, Vcam1, Pamr1, C1s, Tnip3, Serpinb9b, Sult1a1, Bicc1, Gbp2, Msrb3, D0H4S114, Serpinb9, Cyp4a12b, LOC100045065, C3, Glipr2, Ptgs1, Trp53inp1, Gm5077, Ppp2r2b, Tgm2, Spon2, Dmbt1, Ablim3, and Klhl24.

In some embodiments the marker gene is selected from the group consisting of Cxcl15, Hmgcs2, 5330417C22Rik, Cp, Vcam1, Pamr1, C1s, Tnip3, Serpinb9b, Sult1a1, Bicc1, Gbp2, Msrb3, D0H4S114, Serpinb9, Cyp4a12b, LOC100045065, C3, Glipr2, Ptgs1, Trp53inp1, Gm5077, Ppp2r2b, Tgm2, Spon2, Dmbt1, Ablim3, Klhl24, Lbp, Atp2b4, Serpinb6b, Trp53inp2, Sepp1, Pcdhb16, Chst15, Thbs1, Pcdhb19, Ype12, Masp1, Heg1, Cacna1g, Tcn2, Cyp4b1, Cdon, Synpo, Mras, Gpd1, Sesn3, Gpr146, Hexa, A330074K22Rik, Gstt1, Zfp704, Slc7a2, and Stra6.

In some embodiments the marker gene is selected from the group consisting of Cxcl15, Hmgcs2, 5330417C22Rik, Cp, Vcam1, Pamr1, C1s, Tnip3, Serpinb9b, Sult1a1, Bicc1, Gbp2, Msrb3, D0H4S114, Serpinb9, Cyp4a12b, LOC100045065, C3, Glipr2, Ptgs1, Trp53inp1, Gm5077, Ppp2r2b, Tgm2, Spon2, Dmbt1, Ablim3, Klhl24, Lbp, Atp2b4, Serpinb6b, Trp53inp2, Sepp1, Pcdhb16, Chst15, Thbs1, Pcdhb19, Ype12, Masp1, Heg1, Cacna1g, Tcn2, Cyp4b1, Cdon, Synpo, Mras, Gpd1, Sesn3, Gpr146, Hexa, A330074K22Rik, Gstt1, Zfp704, Slc7a2, Stra6, Gda, Sned1, Gpr126, Pcdhb18, Fmo1, Ccl2, Npr2, A930001N09Rik, Sat1, Plscr2, Antxr1, Pcdhb20, Fam20c, Gstm7, Tmie, Ecscr, Tcp11l2, Man1c1, Tnfsf13, Ppargc1a, Rorc, Csrp1, Tgfb2, Clec2d, Zfp3611, Arid5b, Slc40a1, Akr1c18, Creg1, Prg4, I118, Irgm1, C1rb, Srgap1, Aldh6a1, Tifa, Gas6, Glipr1, Fam107a, Pik3ip1, 4933426M11Rik, Dusp16, Sorbs2, Calcoco1, Trf, Xaf1, Gstm2, Cyp4a12a, C1r, Lamb2, Pgcp, Gm4055, Sox4, Wnt4, Ddx58, and Gm14443.

In other embodiments the marker gene is selected from the group consisting of Cxcl15, Hmgcs2, 5330417C22Rik, Cp, Vcam1, Pamr1, C1s, Tnip3, Serpinb9b, Sult1a1, Bicc1, Gbp2, Msrb3, D0H4S114, Serpinb9, Cyp4a12b, LOC100045065, C3, Glipr2, Ptgs1, Trp53inp1, Gm5077, Ppp2r2b, Tgm2, Spon2, Dmbt1, Ablim3, Klhl24, Lbp, Atp2b4, Serpinb6b, Trp53inp2, Sepp1, Pcdhb16, Chst15, Thbs1, Pcdhb19, Ype12, Masp1, Heg1, Cacna1g, Tcn2, Cyp4b1, Cdon, Synpo, Mras, Gpd1, Sesn3, Gpr146, Hexa, A330074K22Rik, Gstt1, Zfp704, Slc7a2, Stra6, Gda, Sned1, Gpr126, Pcdhb18, Fmo1, Ccl2, Npr2, A930001N09Rik, Sat1, Plscr2, Antxr1, Pcdhb20, Fam20c, Gstm7, Tmie, Ecscr, Tcp11l2, Man1c1, Tnfsf13, Ppargc1a, Rorc, Csrp1, Tgfb2, Clec2d, Zfp3611, Arid5b, Slc40a1, Akr1c18, Creg1, Prg4, I118, Irgm1, C1rb, Srgap1, Aldh6a1, Tifa, Gas6, Glipr1, Fam107a, Pik3ip1, 4933426M11Rik, Dusp16, Sorbs2, Calcoco1, Trf, Xaf1, Gstm2, Cyp4a12a, C1r, Lamb2, Pgcp, Gm4055, Sox4, Wnt4, Ddx58, Gm14443, Crot, Sort1, Ece1, Edn1, Dusp1, Rasl11a, Parp8, 5133401N09Rik, Bmf, Pion, 9330159M07Rik, Vnn1, Serinc5, Chsy3, Ppfibp2, Tnfrsf9, 2010011120Rik, Jun, Acot2, Faah, Phyh, Arhgap26, Ype15, Cald1, BC023202, Mxd4, Thra, Nedd9, Tmem140, Il6st, Ctns, Epas1, Timp3, Zfyve1, Itih2, Ephb3, D4Bwg0951e, Igfbp7, Pik3r3, Ccpg1, Akap13, Wfdcl, Bmp4, Abca8b, Prss23, Thrb, Pkd2, LOC381508, Txnip, Tapbp, Fbxo32, Apbb3, Zcwpw1, Pik3r1, Pdzk1ip1, Fosl2, Epdr1, Vwa5a, Gm3932, 2410066E13Rik, Atxn1, Ntn4, Galc, Abcg2, Ptpn14, Mcc, Clec2g, Bco2, Parp3, Crebl2, Fam168a, Ifi2711, Pts, Atp1b1, Lmbrd1, 1700112E06Rik, Zbtb10, Pygb, Dpp7, H19, Lamp2, Sesn1, Malat1, 4833442J19Rik, Adamts9, Irs1, Slc31a2, 9230110C19Rik, Aplp2, Ggh, Cerk, Parp12, Rhou, Zhx1, 1110028C15Rik, Hexb, Foxo3, Fbx120, Idua, Gabarap11, Zfp503, Ahnak2, Clqtnf6, Cd1d1, Nfkbia, Arsk, Prtg, B930095G15Rik, Vasn, 0610040J01Rik, Dynll2, Ptprk, Slc16a4, AW549877, Zfp579, 1810020D17Rik, Slc35f5, Igtp, Rnf144b, Slc16a10, D14Ertd436e, Gm6813, Gm14057, 9230105E10Rik, BC031353, Slc25a23, Rab2b, 2210408F21Rik, Ncoa1, B4galt1, Tgfbr2, Ctso, Gbp1, Ypel1, Stx16, Acox3, Tmem57, and Ahr

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1. Bortezomib inhibits NF-κB signaling and induces apoptosis in murine lung adenocarcinoma cell lines. (1A) reduction of nuclear p65 level by bortezomib. Two KP (KrasLSL-G12D/wt;p53flox/flox) cell lines (KP1 and KP2) were treated with 5 nM bortezomib (BZ) or vehicle control (ctrl) for 24 hours. Nuclear (N) and cytoplasmic (C) fractions of protein lysates were immunoblotted with the indicated antibodies. NEMO and Parp serve as cytoplasmic and nuclear loading controls, respectively. (1B) quantitative PCR analysis of a subset of NF-κB target genes in 5 nM bortezomib (BZ)-treated KP cells at the indicated time points. Vehicle control is set to 1. (1C) apoptosis in KP and KP^{M} (KrasLSL-G12D/wt;p53R127H/−) cells induced by bortezomib. Cells were pre-treated with 5 nM bortezomib for 24 hours, and protein lysates were immunoblotted with CC3 and tubulin antibodies. (1D) quantification of cell death in bortezomib-treated cells. Cells were treated with the indicated concentrations of bortezomib for 24 hours, and dead cells were quantified by Trypan blue staining. Error bars are SD (n=3).

FIG. 2: Bortezomib sensitivity correlates with basal NF-κB activity in KP lung adenocarcinoma cell lines. (2A) relative cell viability in cell lines treated with increasing doses of bortezomib for 24 hours. 3TZ, diamond; LKR13, triangle; KP, solid lines; KPM, dashed lines. Error bars are SD (n=3). (2B) lung cancer cell lines with high basal NF-κB activity were sensitive to bortezomib. NF-κB p65 DNA-binding activity was determined by ELISA from nuclear extracts of KP cell lines and LKR13 cells (set as 1). Relative viability was measured after 5 nM bortezomib treatment, as in 2A.

FIG. 3: Bortezomib induces acute lung tumor regression in KrasLSL-G12D/wt;p53flox/flox (KP) mice, but not in KrasLSL-G12D/wt (K) mice. (3A and 3C) representative microCT images (n=6) of KP (3A) and K (3C) lung tumors prior to pre-treatment (D0) and 4 days (D4) after a single dose of vehicle control or bortezomib. (3 B and 3D) Quantification of tumor volume change (D4 compared with D0) in control- and bortezomib-treated mice. Each bar represents an individual tumor. P<10⁻⁶ in KP mice (3B); P>0.05 in K mice (3D).

FIG. 4: Bortezomib induces apoptosis in KP lung tumors. (4A and 4B) H&E and CC3 immunohistochemistry staining (×200) in vehicle control- and bortezomib-treated KP (4A) and K (4B) lung tumors. (4C and 4D) quantification of CC3-positive cells in bortezomib-treated KP (4C) and K (4D) lung tumors at indicated time points. “0 hour” represents vehicle control-treated tumors. Error bars are SD (n>10 for each time point).

FIG. 5: Bortezomib increases survival in KP mice. (5A and 5B) Kaplan-Meier survival curve of vehicle control and bortezomib-treated KP (5A) and K (5B) mice. Arrows indicate weekly 1 mg/kg bortezomib regimen. (5C) quantification of lung tumor volume of control- and bortezomib-treated KP mice. From 10 weeks after Adeno-Cre infection, lungs were imaged by microCT to measure individual tumor volumes (n=4). Data points represent means of fold change and SD relative to 10 weeks (set to 1). Arrows indicate bortezomib injection. D, CC3 staining (×200) in lung tumors receiving 3 doses of control (“C”) and 1 final dose of bortezomib (“B,” left) or 4 doses of bortezomib (right). Tumors were harvested 48 hours after the last treatment (n=4).

FIG. 6: Imaging bortezomib response and resistance in an orthotopic lung tumor model. (6A) experimental design. Lung cancer cells derived from KP tumors were infected with a retrovirus expressing luciferase and transplanted into immunocompetent recipient mice by tail vein injection. (6B) Kaplan-Meier survival curve of control and bortezomib-treated recipient mice (n=6; P=1.4×10−5). A total of 10,000 cells were transplanted, and treatment started after 35 days. (6C) representative bioluminescence imaging of recipient mice (n=6) treated with vehicle control or bortezomib, as in 6B. D0 refers to the first treatment. Arrows indicate weekly bortezomib regimen.

FIG. 7: The NF-κB inhibitor Bay-117082 leads to lung tumor regression in vivo. (7A) quantitative PCR analysis of NF-κB target gene expression in KP cells treated with 10 μM Bay-117082 for indicated hours. Error bars are SD (n=3). (7B) Bay-117082 treatment leads to lung tumor regression and delays tumor progression in the orthotopic lung tumor model. A total of 50,000 luciferase-tagged KP cells were transplanted into recipient mice (n=6), and treatment was started at 19 days after transplantation (set at D0). Mice were treated with vehicle control or 10 mg/kg Bay-117082 by i.p. injection, and imaged at the indicated time points. Arrows indicate Bay-117082 injections. (7C) Bay-117082 prolongs survival in the KP model (n=6; P=0.008). “i.p. dosing” indicates treatment with 3 doses per week.

FIG. 8: Bortezomib inhibits NF-κB signaling in human NSCLC cells. H2122 (KRASG12C;TP53c176F/Q16L) (8A) and H2009 (KRASG12A;TP53R273L) (8B) cell lines were treated with vehicle control or 10 nM Bortezomib for 24 hours. NF-κB target gene expression was measured by QPCR. Error bars are s.d. (n=3). N.D. denotes not detectable. Mutation information is from the Sanger COSMIC database.

FIG. 9: QPCR analysis of NF-κB target genes expression in murine lung adenocarcinoma cell lines. Values in the 3TZ fibroblast cell line is set to 1. Error bars are s.d. (n=3).

FIG. 10: Bortezomib regimen in (A) KP and (B) K mice.

FIG. 11: Bortezomib reduces tumor volume in a subcutaneous tumor model. 1×10⁶ KP lung cancer cells were injected subcutaneously into NCR nu/nu mice. Tumor volume was quantified by caliper measurements. Treatments started when tumors reached −100 mm³ (set to 1 in tumor volume axis and 0 days in time axis). Arrows indicate Bortezomib (1 mg/kg) or Cisplatin (7 mg/kg) injections. Error bars are s.d (n=4).

FIG. 12: Generating Bortezomib-resistant KP cell lines. (12A) Relative cell viability in cell lines treated with increasing doses of Bortezomib for 24 hours. Cells lines were outgrown from Bortezomib resistant orthotopic tumors (resistant 1&2) or sensitive tumors (sensitive) as in FIG. 6C. Error bars are s.d. (n=3). (12B) Colony formation assay in Bortezomib-resistant cells. 104 cells were plated in 6-well plate and treated with indicated drug concentration. Plates were stained 7 days later with crystal violet solution. Drug-containing medium was refreshed every 4 days.

FIG. 13: Bortezomib-resistant KP cell lines showed down-regulation of NF-κB targets regulating apoptosis and cell cycle upon drug treatment. A representative resistant cell line was treated with control or 5 nM Bortezomib for 24 hours. Gene expression level was measured by QPCR and normalized to control-treated cells (set to 1). Error bars are s.d. (n=3).

FIG. 14: Nuclear NF-κB levels in Bortezomib-resistant and sensitive KP cell lines. (14A) Nuclear (N) and cytoplasmic (C) fractions of protein lysates were immunoblotted with the indicated antibodies. Nemo and Parp serve as cytoplasmic and nuclear loading controls respectively. (14B) Activity of NF-κB in the nuclear extract was measured by ELISA assay. Values in the sensitive cell line is set to 1. Error bars are s.d. (n=3, p>0.05 for all NF-κB subunits).

FIG. 15: Immunofluorescence in Bortezomib-resistant and sensitive KP cell lines. Fixed cells were stained with antibodies for p65 (15A) and p52/p100 (15B) (an antibody recognizing both the p52 and its precursor p100) and Alexa-488 secondary antibody (*). Nucleus were stained with DAPI (#). Images are 400× magnitude.

FIG. 16: Transcriptional profile of NK-1CB targets in Bortezomib-resistant KP cell lines. Gene expression level was quantified by QPCR in four sensitive and four resistant KP cell lines. The average of sensitive cells is set to 1. p values indicate genes significantly different in resistant cells. Error bars are s.d. (n=4).

FIG. 17: Bay-117082 induces cell death In KP cells. (17A) Relative cell viability in cell lines treated with increasing doses of Bay-117082 for 48 hours. (17B) Bay-117082 induces apoptosis in KP cells. Cells were treated with 10IJM Bay-117082 for 48 hours. Protein lysates were immunoblotted with cleaved caspase 3 (CC3) and Tubulin antibodies.

FIG. 18: Bay-117082 induces apoptosis in KP lung tumors. (18A), H&E and cleaved caspase 3 (CC3) immunohistochemistry staining (200×) in vehicle control and Bay-117082 treated KP lung tumors (10 mg/kg, 48 hrs). (18B) Quantification of CC3 positive cells. Error bars are s.d. (p<0.05).

FIG. 19 is a schetaic diagram showing the experimental conditions and a set of graphs showing the results. Two sets of mice were treated with 1 kg/mg IV bortezomib, Kras^{G120};p53^{fl/fl} (high NF-κB, top panel) or KrasL^{St-G12Dwt} (low NF-κB, bottom panel). The graph in the top panel showed rapid tumor progression, apoptosis, and increased survival associated with high NF-κB. The relapsed tumors have high Hmcgs2. The graph on the bottom demonstrates that mice with low NF-κB had no tumor regression, apoptosis and no survival advantage.

FIG. 20 is a blot and graph showing the results of expression in various genes in bortezomib resistant cells. The blot at the top of the page demonstrates that Hmgcs2 is highly expressed in these resistant cells. 429 genes demonstrated a greater than two fold change in expression. The top ten highly expressed genes in the bortezomib resistant cells are shown in the graph at the bottom of FIG. 20.

FIG. 21 is a schematic diagram demonstrating the mechanism through which Hmgcs2 regulates ketogenesis. The gene is highly expressed in kidney and liver and generates ketone bodies. It is induced by fasting, cAMP, fatty acid and repressed by insulin. It is highly expressed in chemoresistant tumors and is co-amplified with PHGDH in human cancer.

FIG. 22 shows a blot and a graph of the results of Hmgsc2 expression in bortezomib resistant cells in vitro. The data demonstrate that Hmgsc2 cDNA increases in these cells. The blot is shown in the left hand panel and the graph is shown in the right hand panel.

FIG. 23 is a set of graphs demonstrating that Hmgsc2 shRNA reduces bortezomib resistance in vitro. The graph in the left hand panel shows the relative amount of Hmgsc2 mRNA in bortezomib resistant cells when the cells are treated with Hmgsc2 shRNA, a control shRNA or insulin (positive control). As shown in the data the control levels of Hmgsc2 are quite high in the control and low in the other samples. The graph on the right hand side of the page shows the relative viability in bortezomib resistant cells at 48 hours treated under the conditions described above (in bortezomib resistant cells when the cells are treated with Hmgsc2 shRNA, a control shRNA or insulin). The shRNA to Hmgsc2 significantly reduced cell viability.

### DETAILED DESCRIPTION

Methods for treating and determining effective therapies for patients having lung cancer are described herein. NF-κB signaling plays an important role in lung cancer development. The invention in some aspects involves the use of NF-κB inhibitory drugs as targeted therapies for lung cancers and in particular embodiments, lung cancers with mutations in Kras and p53 or with activation or enhancement of the NF-κB pathway. It has been discovered that specific inhibition, e.g. by small-molecule inhibitors, of the NF-κB pathway can cause tumor regression. In some instances the specific effects of 2 general NF-κB inhibitors, bortezomib and Bay-117082 are described as examples of NF-κB inhibition, but the invention is not limited to these specific inhibitors, except where otherwise explicitly indicated. It was also discovered that in some instances long-term treatment is associated with acquired resistance to the NF-κB inhibitors. However markers of this resistance have been identified and can be used to determine patient suitability for using NF-κB inhibitor therapy or ending or reducing NF-κB inhibitor therapy.

Although small-molecule compound inhibitors of NF-κB have been proposed as rational single-agent therapies for cancers with aberrant NF-κB activity, most classic NF-κB inhibitors are poorly selective and have known off-target effects (6, 17). It has been discovered that in lung adenocarcinoma cell lines with high NF-κB activity, the proteasome inhibitor bortezomib efficiently reduced nuclear p65, repressed NF-κB target genes, and rapidly induced apoptosis. Bortezomib also induced lung tumor regression and prolonged survival in tumor-bearing Kras LSL-G12D/wt; p53 flox/flox mice but not in KrasLSL-G12D/wt mice. Bortezomib is an FDA-approved first-line treatment for advanced multiple myeloma, a disease with frequent NF-κB pathway activation (18-21). After repeated treatment, initially sensitive lung tumors became resistant to bortezomib. A second NF-κB inhibitor, Bay-117082, showed similar therapeutic efficacy and acquired resistance in mice.

The NF-κB pathway is an emerging cancer drug target (6, 7). The mammalian NF-κB transcription factor family comprises 5 subunits: RELA (p65), RELB, REL (cRel), NF-κB1 (p50 and its precursor p105), and NF-κB2 (p52 and its precursor p100), which form homodimers or heterodimers (8). Two major NF-κB pathways, canonical and alternative, have been well characterized (9). In the canonical pathway, NF-κB (usually composed of a p65-p50 heterodimer) is inhibited through sequestration in the cytoplasm by the inhibitor of κB (IκB) under nonstimulated conditions. IκB is a target of several upstream signaling cascades that activate an IκB kinase (IKK) complex composed of at least 2 kinases, IKKα and IKKβ, and of 1 regulatory subunit, NF-κB essential modulator (NEMO; also called IKKγ). Both IKKα and IKKβ can directly phosphorylate IκB, resulting in its ubiquitination and degradation by the 26S proteasome (7). Once released from IκB, NF-κB becomes active through nuclear translocation and DNA binding. In the alternative pathway, IKKα, activated by NF-κB-inducing kinase, phosphorylates p100, resulting in limited degradation of p100 into p52 by the proteasome, followed by nuclear translocation of the RELB-p52 heterodimer (6).

Mouse models of human cancer are powerful tools to study tumor biology, genetics, and therapies. Previously, mouse models of Eμ-Myc B-cell lymphoma were successfully used to examine the chemotherapy response (25). Similar studies in mouse models of lung cancer have led to new insights into the activity of PI3K inhibitors (26) and cisplatin in vivo (2). Our laboratory has developed an autochthonous mouse model of human lung cancer, in which lung tumors are initiated upon Cre recombinase-mediated activation of a KrasG12D allele. In this case, KrasG12D activation alone (KrasLSL-G12D/wt, K model) generates low-grade adenocarcinomas (27). When combined with the concomitant loss of both p53 alleles (KrasLSL-12D/wt;p53flox/flox, KP model), the mice develop lung tumors with a shorter latency and advanced histopathology (28, 29). These models are thus suitable for evaluating novel targeted small-molecule compounds in a physiological setting.

In one aspect, the invention is useful to screen a population of subjects to identify those that should be treated with an NF-κB inhibitor. The invention is also useful to identify subjects that should be treated with a drug or therapy regimen that does not include an NF-κB inhibitor. These subjects are examined for the presence of a cancer having elevated levels of NF-κB and/or mutations associated with elevated levels of NF-κB such as Kras. The presence of elevated levels of NF-κB or mutations associated therewith are well known by the skilled artisan.

Accordingly, in some embodiments, the invention provides methods for treating a cancer patient including the step of administering to the patient a therapeutically effective amount of a NF-κB inhibitor. An NF-κB inhibitor, as used herein, refers to a compound that can reduce NF-κB signaling in a cancer cell. These inhibitors include but are not limited to small molecule inhibitors, nucleic acid inhibitors and peptide based inhibitors. Small molecule inhibitors include but are not limited to bortezomib and Bay-117082. Other NF-κB inhibitors are well known in the art. “Bortezomib” as used herein refers to ((N-(2-pyrazine) carbonyl-L-phenylalanine-L-leucine boronic acid), or VELACADE® (Millennium Pharmaceuticals) which is a 26S proteasome inhibitor that is approved for use in treating various neoplastic diseases, and especially treatment of relapsed multiple myeloma and mantle cell lymphoma, as well as analogs and variants thereof. Bay-117082 as used herein refers to (E)-3-[(4-methylphenylsulfonyl]-2-propenenitrile, as well as analogs and variants thereof.

(E)-3-[(4-methylphenylsulfonyl]-2-propenenitrile has the structure:

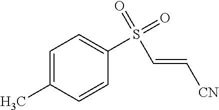

Bortezomib has the structure:

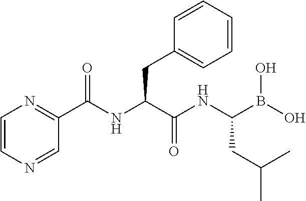 [SMILES: CC(C)C[C@H](NC(=O)[C@H](Cc1ccccc1)NC(=O)c1cnccn1)B(O)O]

The invention also embraces NF-κB inhibitor analogs such as bortezomib or Bay-117082 analogs. Bortezomib or Bay-117082 analogs are chemically modified versions of bortezomib or Bay-117082. In some embodiments, the bortezomib or Bay-117082 analogs have one or more bortezomib or Bay-117082 activities (as described herein), e.g., anti-cancer activity. The one or more activities are preferably present in the bortezomib or Bay-117082 analogs in significant amounts, e.g., at greater than 10%, 20%, 30%, 40%, 50%, 60%, 70%, 80%, or 90% of the activity of bortezomib or Bay-117082, respectively. More preferably, the one or more activities are preferably present in the bortezomib or Bay-117082 analogs at greater than 100%, 110%, 120%, 130%, 140%, 150%, 160%, 170%, 180%, 190%, 200%, or more, of the activity of bortezomib or Bay-117082. The bortezomib or Bay-117082 analogs may not have all of the activities of bortezomib or Bay-117082. However, non-active bortezomib or Bay-117082 analogs, having none of the activities of bortezomib or Bay-117082 in significant amounts, are not useful in the methods of the invention.

According to the invention, useful NF-κB inhibitors include any one or more NF-κB inhibitors including bortezomib and Bay-117082 and/or stereoisomeric forms, or pharmaceutically acceptable acid or base addition salt forms thereof or analogs thereof, in therapeutically effective amounts. The following publications describe useful NF-κB inhibitors such as bortezomib and Bay-117082 and compositions and analogs thereof and therapeutic methods U.S. Pat. Nos. 5,780,454, 6,083,903, 6,297,217, 6,617,317, 6,713,446, 6,747,150, 6,958,319, 7,119,080. The disclosures of these patents are incorporated by reference herein in their entirety for the disclosure of NF-κB inhibitors useful in the methods of the invention.

These different inhibitor compounds and preparations described herein can be administered as a single dose or in several doses administered over a period of time (e.g. chronic administration at regular intervals of time) as described herein. Methods and compositions of the invention include stereoisomeric forms and pharmaceutically acceptable acid or base addition salt forms of the NF-κB inhibitors and NF-κB responsive agents (described below).

In one aspect, the invention is useful for identifying cancers that are responsive to NF-κB treatment. In some embodiments, the level of expression marker shown in Table 1 in a cancer is assayed and the level of expression marker (shown in Table 1) is analyzed to determine the responsiveness of the cancer to NF-κB. For example, the expression level can be compared to one or more reference or threshold levels to determine the responsiveness of the cancer to the therapy. This method is useful for instance for determining whether a cancer is responsive or unresponsive to treatment with a NF-κB inhibitor. A cancer that is responsive to NF-κB inhibitor treatment is one which results in apoptosis of the tumor cells.

A cancer that is “unresponsive to treatment by one or more NF-κB inhibitors”, is a cancer that does not respond to treatment with NF-κB inhibitors. Thus, a subject having such a cancer will not be responsive to treatment with NF-κB inhibitors. While the subject having such a cancer may initially respond to treatment, e.g., the cancer may initially go in remission or further growth of the cancer may initially be suppressed, the cancer may ultimately become resistant to treatment by the NF-κB inhibitors and the cancer will no longer respond to treatment.

Whether a cancer is responsive or non-responsive to NF-κB inhibitors can be determined by examining expression levels of one or more markers in Table 1. In some embodiments the markers are those shown in Table 1 having a Resistant v. Parent FC value of greater than 1.0, 1.5, 2.0, 2.5, 3.0, or 3.5.

TABLE 1

| Gene name | Resistant_v_Parent_FC (Log2) | Parent_v_Resistant_Pval_Anova |
|---|---|---|
| Cxcl15 | 7.02 | 0.0029 |
| Hmgcs2 | 5.72 | 0.0011 |
| 5330417C22Rik | 4.27 | 0.0148 |
| Cp | 3.96 | 0.0000 |
| Vcam1 | 3.96 | 0.0001 |
| Pamr1 | 3.85 | 0.0322 |
| C1s | 3.62 | 0.0118 |
| Tnip3 | 3.54 | 0.0007 |
| Serpinb9b | 3.27 | 0.0007 |
| Sult1a1 | 3.15 | 0.0072 |
| Bicc1 | 3.09 | 0.0011 |
| Gbp2 | 3.08 | 0.0031 |
| Msrb3 | 3.00 | 0.0254 |
| D0H4S114 | 2.98 | 0.0839 |
| Serpinb9 | 2.96 | 0.0042 |
| Cyp4a12b | 2.91 | 0.0700 |
| LOC100045065 | 2.86 | 0.0184 |
| C3 | 2.85 | 0.0280 |
| Glipr2 | 2.80 | 0.0624 |
| Ptgs1 | 2.76 | 0.0810 |
| Trp53inp1 | 2.73 | 0.0052 |
| Gm5077 | 2.62 | 0.0188 |
| Ppp2r2b | 2.62 | 0.0456 |
| Tgm2 | 2.61 | 0.0209 |
| Spon2 | 2.60 | 0.0430 |
| Dmbt1 | 2.53 | 0.0668 |
| Ablim3 | 2.51 | 0.0924 |
| Klh124 | 2.50 | 0.0028 |
| Lbp | 2.46 | 0.0032 |
| Atp2b4 | 2.41 | 0.0863 |
| Serpinb6b | 2.39 | 0.0004 |
| Trp53inp2 | 2.38 | 0.0207 |
| Sepp1 | 2.38 | 0.0013 |
| Pcdhb16 | 2.35 | 0.0336 |
| Chst15 | 2.35 | 0.0055 |
| Thbs1 | 2.29 | 0.0537 |
| Pcdhb19 | 2.28 | 0.0246 |
| Ypel2 | 2.24 | 0.0223 |
| Masp1 | 2.23 | 0.0552 |
| Heg1 | 2.21 | 0.0623 |
| Cacna1g | 2.18 | 0.0518 |
| Tcn2 | 2.18 | 0.0287 |
| Cyp4b1 | 2.17 | 0.0014 |
| Cdon | 2.14 | 0.0407 |
| Synpo | 2.11 | 0.0797 |
| Mras | 2.10 | 0.0012 |
| Gpd1 | 2.09 | 0.0003 |
| Sesn3 | 2.08 | 0.0126 |
| Gpr146 | 2.06 | 0.0454 |
| Hexa | 2.03 | 0.0078 |
| A330074K22Rik | 2.03 | 0.0663 |
| Gstt1 | 2.02 | 0.0228 |
| Zfp704 | 2.01 | 0.0129 |
| Slc7a2 | 2.00 | 0.0829 |
| Stra6 | 2.00 | 0.0006 |
| Gda | 1.99 | 0.0166 |
| Sned1 | 1.99 | 0.0166 |
| Gpr126 | 1.99 | 0.0111 |
| Pcdhb18 | 1.98 | 0.0930 |
| Fmo1 | 1.97 | 0.0593 |
| Cc12 | 1.92 | 0.0365 |
| Npr2 | 1.91 | 0.0893 |
| A930001N09Rik | 1.91 | 0.0013 |
| Sat1 | 1.90 | 0.0006 |
| Plscr2 | 1.90 | 0.0142 |
| Antxr1 | 1.89 | 0.0003 |
| Pcdhb20 | 1.89 | 0.0120 |
| Fam20c | 1.89 | 0.0663 |
| Gstm7 | 1.87 | 0.0680 |
| Tmie | 1.85 | 0.0027 |
| Ecscr | 1.84 | 0.0094 |
| Tcp1112 | 1.81 | 0.0015 |
| Man1c1 | 1.81 | 0.0675 |
| Tnfsf13 | 1.80 | 0.0017 |
| Ppargc1a | 1.79 | 0.0110 |
| Rorc | 1.79 | 0.0864 |
| Csrp1 | 1.76 | 0.0937 |
| Tgfb2 | 1.76 | 0.0740 |
| Clec2d | 1.76 | 0.0211 |
| Zfp3611 | 1.76 | 0.0010 |
| Arid5b | 1.74 | 0.0099 |
| Slc40a1 | 1.74 | 0.0040 |
| Akr1c18 | 1.72 | 0.0923 |
| Creg1 | 1.72 | 0.0163 |
| Prg4 | 1.72 | 0.0085 |
| I118 | 1.69 | 0.0086 |
| Irgm1 | 1.69 | 0.0411 |
| C1rb | 1.68 | 0.0012 |
| Srgap1 | 1.68 | 0.0008 |
| Aldh6a1 | 1.66 | 0.0777 |
| Tifa | 1.66 | 0.0204 |
| Gas6 | 1.66 | 0.0979 |
| Glipr1 | 1.65 | 0.0341 |
| Fam107a | 1.65 | 0.0547 |
| Pik3ip1 | 1.64 | 0.0660 |
| 4933426M11Rik | 1.64 | 0.0009 |
| Dusp16 | 1.63 | 0.0190 |
| Sorbs2 | 1.63 | 0.0374 |
| Calcoco1 | 1.62 | 0.0214 |
| Trf | 1.61 | 0.0035 |
| Xaf1 | 1.60 | 0.0828 |
| Gstm2 | 1.60 | 0.0243 |
| Cyp4a12a | 1.57 | 0.0757 |
| C1r | 1.56 | 0.0005 |
| Lamb2 | 1.56 | 0.0271 |
| Pgcp | 1.55 | 0.0061 |
| Gm4055 | 1.54 | 0.0158 |
| Sox4 | 1.53 | 0.0361 |
| Wnt4 | 1.52 | 0.0079 |
| Ddx58 | 1.51 | 0.0280 |
| Gm14443 | 1.50 | 0.0447 |
| Crot | 1.49 | 0.0189 |
| Sort1 | 1.48 | 0.0513 |
| Ece1 | 1.48 | 0.0652 |
| Edn1 | 1.48 | 0.0402 |
| Dusp1 | 1.48 | 0.0417 |
| Rasl11a | 1.48 | 0.0190 |
| Parp8 | 1.47 | 0.0030 |
| 5133401N09Rik | 1.47 | 0.0044 |
| Bmf | 1.45 | 0.0115 |
| Pion | 1.45 | 0.0232 |
| 9330159M07Rik | 1.45 | 0.0408 |
| Vnn1 | 1.44 | 0.0068 |
| Serinc5 | 1.44 | 0.0348 |
| Chsy3 | 1.43 | 0.0268 |
| Ppfibp2 | 1.43 | 0.0096 |
| Tnfrsf9 | 1.42 | 0.0870 |
| 2010011I20Rik | 1.42 | 0.0866 |
| Jun | 1.42 | 0.0489 |
| Acot2 | 1.41 | 0.0034 |
| Faah | 1.41 | 0.0792 |
| Phyh | 1.40 | 0.0100 |
| Arhgap26 | 1.39 | 0.0267 |
| Ypel5 | 1.39 | 0.0823 |
| Cald1 | 1.39 | 0.0462 |
| BC023202 | 1.38 | 0.0320 |
| Mxd4 | 1.38 | 0.0359 |
| Thra | 1.38 | 0.0692 |
| Nedd9 | 1.37 | 0.0178 |
| Tmem140 | 1.37 | 0.0477 |
| Il6st | 1.36 | 0.0052 |
| Ctns | 1.36 | 0.0224 |
| Epas1 | 1.36 | 0.0926 |
| Timp3 | 1.35 | 0.0382 |
| Zfyve1 | 1.35 | 0.0263 |
| Itih2 | 1.35 | 0.0254 |
| Ephb3 | 1.33 | 0.0281 |
| D4Bwg0951e | 1.33 | 0.0254 |
| Igfbp7 | 1.33 | 0.0684 |
| Pik3r3 | 1.32 | 0.0482 |
| Ccpg1 | 1.32 | 0.0002 |
| Akap13 | 1.32 | 0.0353 |
| Wfdc1 | 1.32 | 0.0569 |
| Bmp4 | 1.32 | 0.0511 |
| Abca8b | 1.32 | 0.0890 |
| Prss23 | 1.31 | 0.0070 |
| Thrb | 1.30 | 0.0441 |
| Pkd2 | 1.30 | 0.0609 |
| LOC381508 | 1.30 | 0.0503 |
| Txnip | 1.29 | 0.0895 |
| Tapbp | 1.29 | 0.0631 |
| Fbxo32 | 1.29 | 0.0349 |
| Apbb3 | 1.26 | 0.0231 |
| Zcwpw1 | 1.26 | 0.0033 |
| Pik3r1 | 1.25 | 0.0734 |
| Pdzk1ip1 | 1.24 | 0.0151 |
| Fos12 | 1.24 | 0.0977 |
| Epdr1 | 1.23 | 0.0009 |
| Vwa5a | 1.23 | 0.0669 |
| Gm3932 | 1.23 | 0.0596 |
| 2410066E13Rik | 1.22 | 0.0437 |
| Atxn1 | 1.22 | 0.0167 |
| Ntn4 | 1.22 | 0.0555 |
| Galc | 1.22 | 0.0558 |
| Abcg2 | 1.21 | 0.0314 |
| Ptpn14 | 1.20 | 0.0735 |
| Mcc | 1.18 | 0.0080 |
| Clec2g | 1.18 | 0.0070 |
| Bco2 | 1.17 | 0.0052 |
| Parp3 | 1.17 | 0.0022 |
| Creb12 | 1.17 | 0.0027 |
| Fam168a | 1.17 | 0.0950 |
| Ifi2711 | 1.17 | 0.0053 |
| Pts | 1.16 | 0.0286 |
| Atp1b1 | 1.16 | 0.0222 |
| Lmbrd1 | 1.16 | 0.0254 |
| 1700112E06Rik | 1.16 | 0.0218 |
| Zbtb10 | 1.16 | 0.0187 |
| Pygb | 1.16 | 0.0405 |
| Dpp7 | 1.15 | 0.0406 |
| H19 | 1.15 | 0.0875 |
| Lamp2 | 1.14 | 0.0025 |
| Sesn1 | 1.14 | 0.0214 |
| Malat1 | 1.14 | 0.0453 |
| 4833442J19Rik | 1.14 | 0.0052 |
| Adamts9 | 1.14 | 0.0340 |
| Irs1 | 1.14 | 0.0747 |
| Slc31a2 | 1.13 | 0.0213 |
| 9230110C19Rik | 1.13 | 0.0619 |
| Aplp2 | 1.12 | 0.0164 |
| Ggh | 1.12 | 0.0060 |
| Cerk | 1.12 | 0.0294 |
| Parp12 | 1.12 | 0.0453 |
| Rhou | 1.11 | 0.0024 |
| Zhx1 | 1.11 | 0.0999 |
| 1110028C15Rik | 1.11 | 0.0126 |
| Hexb | 1.10 | 0.0341 |
| Foxo3 | 1.10 | 0.0964 |
| Fbx120 | 1.10 | 0.0329 |
| Idua | 1.09 | 0.0071 |
| Gabarap11 | 1.09 | 0.0770 |
| Zfp503 | 1.09 | 0.0615 |
| Ahnak2 | 1.09 | 0.0050 |
| Clqtnf6 | 1.09 | 0.0589 |
| Cdld1 | 1.08 | 0.0164 |
| Nfkbia | 1.08 | 0.0007 |
| Arsk | 1.08 | 0.0088 |
| Prtg | 1.08 | 0.0845 |
| B930095G15Rik | 1.08 | 0.0703 |
| Vasn | 1.08 | 0.0821 |
| 0610040J01Rik | 1.07 | 0.0059 |
| Dynll2 | 1.07 | 0.0029 |
| Ptprk | 1.07 | 0.0058 |
| S1c16a4 | 1.07 | 0.0720 |
| AW549877 | 1.07 | 0.0683 |
| Zfp579 | 1.06 | 0.0339 |
| 1810020D17Rik | 1.06 | 0.0199 |
| Slc35f5 | 1.06 | 0.0216 |
| Igtp | 1.05 | 0.0328 |
| Rnf144b | 1.05 | 0.0371 |
| Slc16a10 | 1.05 | 0.0006 |
| D14Ertd436e | 1.04 | 0.0136 |
| Gm6813 | 1.04 | 0.0099 |
| Gm14057 | 1.04 | 0.0920 |
| 9230105E10Rik | 1.03 | 0.0107 |
| BC031353 | 1.03 | 0.0095 |
| S1c25a23 | 1.03 | 0.0893 |
| Rab2b | 1.03 | 0.0267 |
| 2210408F21Rik | 1.03 | 0.0345 |
| Ncoa1 | 1.02 | 0.0766 |
| B4galt1 | 1.02 | 0.0184 |
| Tgfbr2 | 1.02 | 0.0511 |
| Ctso | 1.02 | 0.0173 |
| Gbp1 | 1.01 | 0.0524 |
| Ype11 | 1.01 | 0.0333 |
| Stx16 | 1.01 | 0.0004 |
| Acox3 | 1.01 | 0.0624 |
| Tmem57 | 1.01 | 0.0359 |
| Ahr | 1.01 | 0.0622 |
| Cables1 | 1.00 | 0.0799 |
| Pawr | 1.00 | 0.0889 |
| Recq14 | −1.00 | 0.0491 |
| Krt7 | −1.00 | 0.0704 |
| Qtrtd1 | −1.00 | 0.0739 |
| Ddx19b | −1.01 | 0.0109 |
| Ltv1 | −1.01 | 0.0075 |
| Npm3 | −1.01 | 0.0108 |
| 2700094K13Rik | −1.01 | 0.0630 |
| 1500011K16Rik | −1.01 | 0.0380 |
| 2810433K01Rik | −1.01 | 0.0281 |
| Snrpal | −1.01 | 0.0157 |
| Rin1 | −1.02 | 0.0949 |
| Timm8al | −1.02 | 0.0317 |
| Ak311 | −1.03 | 0.0321 |
| Ddx39 | −1.03 | 0.0064 |
| Nop56 | −1.03 | 0.0298 |
| Klra2 | −1.04 | 0.0821 |
| Nat10 | −1.04 | 0.0139 |
| Zdhhc2 | −1.04 | 0.0724 |
| Arhgap19 | −1.04 | 0.0710 |
| Fmn11 | −1.05 | 0.0174 |
| Pold1 | −1.05 | 0.0530 |
| Wbscr16 | −1.05 | 0.0055 |
| Wdr74 | −1.06 | 0.0112 |
| Prim1 | −1.06 | 0.0757 |
| Timm50 | −1.06 | 0.0488 |
| Hirip3 | −1.06 | 0.0787 |
| Sema6b | −1.07 | 0.0331 |
| Itgb7 | −1.07 | 0.0201 |
| Dyrk3 | −1.07 | 0.0032 |
| Tuba4a | −1.07 | 0.0106 |
| My17 | −1.07 | 0.0663 |
| Gins1 | −1.07 | 0.0212 |
| Cubn | −1.08 | 0.0420 |
| Nasp | −1.08 | 0.0717 |
| Mnd1 | −1.09 | 0.0582 |
| Pmf1 | −1.09 | 0.0256 |
| Psat1 | −1.10 | 0.0242 |
| Pop1 | −1.10 | 0.0440 |
| Nrg2 | −1.10 | 0.0356 |
| Snai3 | −1.10 | 0.0182 |
| Tmem138 | −1.11 | 0.0053 |
| Mars | −1.11 | 0.0299 |
| S1c29a2 | −1.11 | 0.0463 |
| Fos11 | −1.11 | 0.0031 |
| Ccdc92 | −1.11 | 0.0148 |
| Mtbp | −1.11 | 0.0257 |
| Tnfsf9 | −1.11 | 0.0591 |
| Timm13 | −1.12 | 0.0024 |
| G630025P09Rik | −1.12 | 0.0112 |
| Thoc4 | −1.13 | 0.0246 |
| E2f4 | −1.13 | 0.0028 |
| Ahcy | −1.13 | 0.0717 |
| Cdc451 | −1.13 | 0.0499 |
| Tbc1d2 | −1.13 | 0.0423 |
| Zdhhc21 | −1.13 | 0.0859 |
| Tnnt2 | −1.13 | 0.0052 |
| Prr11 | −1.13 | 0.0892 |
| 5730408K05Rik | −1.14 | 0.0116 |
| Spag5 | −1.14 | 0.0873 |
| 1600029D21Rik | −1.14 | 0.0130 |
| E130303B06Rik | −1.14 | 0.0205 |
| Sox13 | −1.15 | 0.0077 |
| Chd11 | −1.15 | 0.0749 |
| Cdc20 | −1.15 | 0.0958 |
| Lyar | −1.15 | 0.0525 |
| Cgref1 | −1.16 | 0.0224 |
| Cad | −1.16 | 0.0342 |
| LOC433316 | −1.16 | 0.0117 |
| Mcm5 | −1.16 | 0.0948 |
| Aurka | −1.17 | 0.0772 |
| Cdca2 | −1.17 | 0.0520 |
| Cars | −1.17 | 0.0945 |
| Fxn | −1.18 | 0.0372 |
| 6720463M24Rik | −1.18 | 0.0333 |
| Incenp | −1.19 | 0.0083 |
| Dusp5 | −1.20 | 0.0315 |
| Xylb | −1.20 | 0.0421 |
| Eepd1 | −1.20 | 0.0957 |
| Grwd1 | −1.20 | 0.0094 |
| Exo1 | −1.20 | 0.0859 |
| Sh2d1b1 | −1.20 | 0.0058 |
| Kif22 | −1.21 | 0.0923 |
| 2810026P18Rik | −1.21 | 0.0821 |
| Eif5a | −1.21 | 0.0011 |
| Melk | −1.22 | 0.0906 |
| Gpsm2 | −1.22 | 0.0026 |
| Cdc25c | −1.23 | 0.0462 |
| Pola2 | −1.23 | 0.0024 |
| Wdr54 | −1.23 | 0.0790 |
| Opn3 | −1.24 | 0.0145 |
| Rangrf | −1.24 | 0.0092 |
| Dna2 | −1.25 | 0.0398 |
| Cib1 | −1.25 | 0.0695 |
| Ndrg2 | −1.26 | 0.0617 |
| Dus41 | −1.26 | 0.0807 |
| Asns | −1.27 | 0.0612 |
| Ank1 | −1.28 | 0.0124 |
| Tk1 | −1.28 | 0.0325 |
| Qk | −1.28 | 0.0064 |
| Mfsd2 | −1.28 | 0.0382 |
| Lig1 | −1.29 | 0.0644 |
| Prmt7 | −1.29 | 0.0558 |
| Mthfd11 | −1.29 | 0.0588 |
| Ngf | −1.29 | 0.0248 |
| Snhg7 | −1.30 | 0.0717 |
| Tb13 | −1.30 | 0.0203 |
| Dhps | −1.30 | 0.0267 |
| Tacc3 | −1.30 | 0.0356 |
| Eme1 | −1.30 | 0.0150 |
| Zdhhc23 | −1.32 | 0.0260 |
| Hpdl | −1.33 | 0.0003 |
| Npm3-ps1 | −1.33 | 0.0042 |
| Suv39h2 | −1.34 | 0.0709 |
| Spint1 | −1.34 | 0.0581 |
| Hist3h2ba | −1.35 | 0.0076 |
| Prodh | −1.35 | 0.0232 |
| Marveld2 | −1.36 | 0.0946 |
| Rps12 | −1.37 | 0.0399 |
| Ccdc124 | −1.38 | 0.0363 |
| Cth | −1.39 | 0.0337 |
| Tmem163 | −1.39 | 0.0839 |
| Mtmr7 | −1.39 | 0.0786 |
| Gtlf3a | −1.39 | 0.0318 |
| Trip13 | −1.39 | 0.0524 |
| Hmgb2 | −1.39 | 0.0625 |
| Acyl | −1.40 | 0.0523 |
| Dcaf15 | −1.41 | 0.0102 |
| Calcrl | −1.41 | 0.0201 |
| Nup62 | −1.43 | 0.0722 |
| 2610019E17Rik | −1.43 | 0.0167 |
| Fam84a | −1.43 | 0.0610 |
| Nup93 | −1.43 | 0.0037 |
| Fancd2 | −1.45 | 0.0519 |
| Cdca3 | −1.45 | 0.0423 |
| BC030867 | −1.48 | 0.0807 |
| Hist2h4 | −1.49 | 0.0908 |
| Dus21 | −1.49 | 0.0179 |
| Acsbg1 | −1.50 | 0.0686 |
| Mlf1ip | −1.50 | 0.0563 |
| Ccne1 | −1.51 | 0.0160 |
| Cdca5 | −1.52 | 0.0120 |
| Tspan1 | −1.53 | 0.0880 |
| Ptprn | −1.57 | 0.0083 |
| Fanca | −1.58 | 0.0044 |
| Btbd11 | −1.60 | 0.0113 |
| Nme2 | −1.60 | 0.0015 |
| Rps6ka4 | −1.61 | 0.0021 |
| Brca2 | −1.62 | 0.0574 |
| Brip1 | −1.63 | 0.0820 |
| Gpx2 | −1.64 | 0.0380 |
| Fhst4h4 | −1.70 | 0.0553 |
| S1c7a5 | −1.73 | 0.0427 |
| Nup210 | −1.73 | 0.0711 |
| Vcan | −1.73 | 0.0482 |
| Rrp12 | −1.74 | 0.0027 |
| Setd6 | −1.76 | 0.0017 |
| Ckap2 | −1.78 | 0.0545 |
| Gcsh | −1.78 | 0.0010 |
| Pacrg | −1.79 | 0.0439 |
| Mthfd2 | −1.89 | 0.0734 |
| Mrc1 | −1.92 | 0.0727 |
| Adam8 | −1.94 | 0.0066 |
| Myb12 | −1.97 | 0.0635 |
| Cep55 | −2.00 | 0.0282 |
| Ttc9 | −2.00 | 0.0987 |
| Gm129 | −2.06 | 0.0577 |
| 2310002L13Rik | −2.17 | 0.0194 |
| Has3 | −2.26 | 0.0737 |
| Casc5 | −2.30 | 0.0823 |
| S1c7a11 | −2.44 | 0.0451 |
| Phlda2 | −2.51 | 0.0163 |
| Nptx1 | −2.57 | 0.0241 |
| C3ar1 | −2.63 | 0.0890 |
| Kcnk1 | −2.86 | 0.0050 |
| Apoc2 | −3.03 | 0.0680 |
| Trpa1 | −3.17 | 0.0849 |
| Serpinb5 | −3.20 | 0.0067 |
| Acan | −3.48 | 0.0299 |

In another aspect, the invention provides a combination therapy that includes an NF-κB inhibitor and a NF-κB responsive agent. An NF-κB responsive agent is a compound that is useful for treating a cancer that is unresponsive to treatment by one or more NF-κB inhibitors as assessed by markers of Table 1. Thus, according to the invention, this combination therapy is particularly useful to treat cancers that express one or more of the markers of Table 1, such as HMG-CoA synthase 2. In other embodiments, the invention provides methods for treating a cancer patient including the step of administering to the patient a therapeutically effective amount of a NF-κB inhibitor compound and a therapeutically effective amount of a NF-κB responsive agent or a therapeutic preparation of these compounds. The NF-κB inhibitor compound can be administered along with, before, or after the NF-κB responsive agent compound. In some embodiments, the NF-κB inhibitor compound and NF-κB responsive agent compounds can be formulated into a single therapeutic preparation.

In another aspect, the invention provides methods for identifying patients that should be treated with a combination of an NF-κB inhibitor and a NF-κB responsive agent. In some embodiments, the level of expression marker shown in Table 1 in a cancer is determined, and a patient is identified as a candidate for a combination therapy if the expression marker shown in Table 1 level is above one or more reference or threshold levels.

The methods disclosed herein typically involve determining expression levels of at least one marker gene in a clinical sample obtained from an individual. The methods may involve determining expression levels of at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more marker genes in a clinical sample obtained from an individual. The methods may involve determining expression levels of 1 to 10, 10 to 20, 20 to 30, 30 to 40, 40 to 50, 50 to 60, 60 to 70, 70 to 80, 80 to 90, or 90 to 100 marker genes in a clinical sample obtained from an individual. The methods may involve determining expression levels of about 10, about 20, about 30, about 35, about 40, about 50, about 60, about 70, about 80, about 85, about 90, about 100, or more marker genes in a clinical sample obtained from an individual.

An expression level determining system may be used in the methods. The term “expression level determining system”, as used herein, refers to a set of components, equipment, and/or reagents, for determining the expression level of a gene in a sample. The expression level of a marker gene may be determined as the level of an RNA encoded by the gene, in which case, the expression level determining system may comprise components useful for determining levels of nucleic acids. The expression level determining system may comprises, for example, hybridization-based assay components, and related equipment and reagents, for determining the level of the RNA in the clinical sample. Hybridization-based assays are well known in the art and include, but are not limited to, oligonucleotide array assays (e.g., microarray assays), cDNA array assays, oligonucleotide conjugated bead assays (e.g., Multiplex Bead-based Luminex® Assays), molecular inversion probe assay, serial analysis of gene expression (SAGE) assay, RNase Protein Assay, northern blot assay, an in situ hybridization assay, and an RT-PCR assay. Multiplex systems, such as oligonucleotide arrays or bead-based nucleic acid assay systems are particularly useful for evaluating levels of a plurality of nucleic acids in simultaneously. RNA-Seq (mRNA sequencing using Ultra High throughput or Next Generation Sequencing) may also be used to determine expression levels. Other appropriate methods for determining levels of nucleic acids will be apparent to the skilled artisan.

The expression level of marker gene may be determined as the level of a protein encoded by the gene, in which case, the expression level determining system may comprise components useful for determining levels of proteins. The expression level determining system may comprise, for example, antibody-based assay components, and related equipment and reagents, for determining the level of the protein in the clinical sample. Antibody-based assays are well known in the art and include, but are not limited to, antibody array assays, antibody conjugated-bead assays, enzyme-linked immunosorbent (ELISA) assays, immunofluorescence microscopy assays, and immunoblot assays. Other methods for determining protein levels include mass spectroscopy, spectrophotometry, and enzymatic assays. Still other appropriate methods for determining levels of proteins will be apparent to the skilled artisan.

A “NF-κB responsive agent” as used herein refers to an inhibitor of a compound that induces NF-κB resistance, such as the compounds listed in Table 1. Thus, NF-κB responsive agent include but are not limited to inhibitors of the compounds listed in Table 1. A preferred NF-κB responsive agent is an HMG-CoA synthetase 2 inhibitor. Inhibitors of HMG-CoA synthetase 2 are known in the art and include, for instance, inhibitory nucleic acids.

In some embodiments the NF-κB responsive gent is an inhibitor of a compound selected from the group consisting of Cxcl15, Hmgcs2, 5330417C22Rik, Cp, Vcam1, Pamr1, C1s, Tnip3, Serpinb9b, Sult1a1, Bicc1, Gbp2, and Msrb3.

In some embodiments the NF-κB responsive gent is an inhibitor of a compound selected from the group consisting of Cxcl15, Hmgcs2, 5330417C22Rik, Cp, Vcam1, Pamr1, C1s, Tnip3, Serpinb9b, Sult1a1, Bicc1, Gbp2, Msrb3, D0H4S114, Serpinb9, Cyp4a12b, LOC100045065, C3, Glipr2, Ptgs1, Trp53inp1, Gm5077, Ppp2r2b, Tgm2, Spon2, Dmbt1, Ablim3, and Klhl24.

In some embodiments the NF-κB responsive gent is an inhibitor of a compound selected from the group consisting of Cxcl15, Hmgcs2, 5330417C22Rik, Cp, Vcam1, Pamr1, C1s, Tnip3, Serpinb9b, Sult1a1, Bicc1, Gbp2, Msrb3, D0H4S114, Serpinb9, Cyp4a12b, LOC100045065, C3, Glipr2, Ptgs1, Trp53inp1, Gm5077, Ppp2r2b, Tgm2, Spon2, Dmbt1, Ablim3, Klhl24, Lbp, Atp2b4, Serpinb6b, Trp53inp2, Sepp1, Pcdhb16, Chst15, Thbs1, Pcdhb19, Ype12, Masp1, Heg1, Cacna1g, Tcn2, Cyp4b1, Cdon, Synpo, Mras, Gpd1, Sesn3, Gpr146, Hexa, A330074K22Rik, Gstt1, Zfp704, Slc7a2, and Stra6.

In some embodiments the NF-κB responsive gent is an inhibitor of a compound selected from the group consisting of Cxcl15, Hmgcs2, 5330417C22Rik, Cp, Vcam1, Pamr1, C1s, Tnip3, Serpinb9b, Sult1a1, Bicc1, Gbp2, Msrb3, D0H4S114, Serpinb9, Cyp4a12b, LOC100045065, C3, Glipr2, Ptgs1, Trp53inp1, Gm5077, Ppp2r2b, Tgm2, Spon2, Dmbt1, Ablim3, Klhl24, Lbp, Atp2b4, Serpinb6b, Trp53inp2, Sepp1, Pcdhb16, Chst15, Thbs1, Pcdhb19, Ype12, Masp1, Heg1, Cacna1g, Tcn2, Cyp4b1, Cdon, Synpo, Mras, Gpd1, Sesn3, Gpr146, Hexa, A330074K22Rik, Gstt1, Zfp704, Slc7a2, Stra6, Gda, Sned1, Gpr126, Pcdhb18, Fmo1, Ccl2, Npr2, A930001N09Rik, Sat1, Plscr2, Antxr1, Pcdhb20, Fam20c, Gstm7, Tmie, Ecscr, Tcp11l2, Man1c1, Tnfsf13, Ppargc1a, Rorc, Csrp1, Tgfb2, Clec2d, Zfp3611, Arid5b, Slc40a1, Akr1c18, Creg1, Prg4, I118, Irgm1, C1rb, Srgap1, Aldh6a1, Tifa, Gas6, Glipr1, Fam107a, Pik3ip1, 4933426M11Rik, Dusp16, Sorbs2, Calcoco1, Trf, Xaf1, Gstm2, Cyp4a12a, C1r, Lamb2, Pgcp, Gm4055, Sox4, Wnt4, Ddx58, and Gm14443.

In other embodiments the NF-κB responsive gent is an inhibitor of a compound selected from the group consisting of Cxcl15, Hmgcs2, 5330417C22Rik, Cp, Vcam1, Pamr1, C1s, Tnip3, Serpinb9b, Sult1a1, Bicc1, Gbp2, Msrb3, D0H4S114, Serpinb9, Cyp4a12b, LOC100045065, C3, Glipr2, Ptgs1, Trp53inp1, Gm5077, Ppp2r2b, Tgm2, Spon2, Dmbt1, Ablim3, Klhl24, Lbp, Atp2b4, Serpinb6b, Trp53inp2, Sepp1, Pcdhb16, Chst15, Thbs1, Pcdhb19, Ype12, Masp1, Heg1, Cacna1g, Tcn2, Cyp4b1, Cdon, Synpo, Mras, Gpd1, Sesn3, Gpr146, Hexa, A330074K22Rik, Gstt1, Zfp704, Slc7a2, Stra6, Gda, Sned1, Gpr126, Pcdhb18, Fmo1, Ccl2, Npr2, A930001N09Rik, Sat1, Plscr2, Antxr1, Pcdhb20, Fam20c, Gstm7, Tmie, Ecscr, Tcp11l2, Man1c1, Tnfsf13, Ppargc1a, Rorc, Csrp1, Tgfb2, Clec2d, Zfp3611, Arid5b, Slc40a1, Akr1c18, Creg1, Prg4, I118, Irgm1, C1rb, Srgap1, Aldh6a1, Tifa, Gas6, Glipr1, Fam107a, Pik3ip1, 4933426M11Rik, Dusp16, Sorbs2, Calcoco1, Trf, Xaf1, Gstm2, Cyp4a12a, C1r, Lamb2, Pgcp, Gm4055, Sox4, Wnt4, Ddx58, Gm14443, Crot, Sort1, Ece1, Edn1, Dusp1, Rasl11a, Parp8, 5133401N09Rik, Bmf, Pion, 9330159M07Rik, Vnn1, Serinc5, Chsy3, Ppfibp2, Tnfrsf9, 2010011120Rik, Jun, Acot2, Faah, Phyh, Arhgap26, Ype15, Cald1, BC023202, Mxd4, Thra, Nedd9, Tmem140, Il6st, Ctns, Epas1, Timp3, Zfyve1, Itih2, Ephb3, D4Bwg0951e, Igfbp7, Pik3r3, Ccpg1, Akap13, Wfdcl, Bmp4, Abca8b, Prss23, Thrb, Pkd2, LOC381508, Txnip, Tapbp, Fbxo32, Apbb3, Zcwpw1, Pik3r1, Pdzk1ip1, Fosl2, Epdr1, Vwa5a, Gm3932, 2410066E13Rik, Atxn1, Ntn4, Galc, Abcg2, Ptpn14, Mcc, Clec2g, Bco2, Parp3, Crebl2, Fam168a, Ifi2711, Pts, Atp1b1, Lmbrd1, 1700112E06Rik, Zbtb10, Pygb, Dpp7, H19, Lamp2, Sesn1, Malat1, 4833442J19Rik, Adamts9, Irs1, Slc31a2, 9230110C19Rik, Aplp2, Ggh, Cerk, Parp12, Rhou, Zhx1, 1110028C15Rik, Hexb, Foxo3, Fbx120, Idua, Gabarap11, Zfp503, Ahnak2, Clqtnf6, Cd1d1, Nfkbia, Arsk, Prtg, B930095G15Rik, Vasn, 0610040J01Rik, Dynll2, Ptprk, Slc16a4, AW549877, Zfp579, 1810020D17Rik, S1c35f5, Igtp, Rnf144b, Slc16a10, D14Ertd436e, Gm6813, Gm14057, 9230105E10Rik, BC031353, Slc25a23, Rab2b, 2210408F21Rik, Ncoa1, B4galt1, Tgfbr2, Ctso, Gbp1, Ypel1, Stx16, Acox3, Tmem57, and Ahr.

An HMG-CoA synthetase 2 inhibitory nucleic acid causes gene knockdown. HMG-CoA synthetase 2 gene is known in the art, i.e., NM_{—}005518 at chr1:120290619-120311555; GENE symbol=HMGCS2, and protein sequence in uniprot P54868). Various strategies for gene knockdown known in the art can be used to inhibit gene expression (e.g., expression of HMG-CoA synthetase 2 as well as the other genes listed in Table 1). For example, gene knockdown strategies may be used that make use of RNA interference (RNAi) and/or microRNA (miRNA) pathways including small interfering RNA (siRNA), short hairpin RNA (shRNA), double-stranded RNA (dsRNA), miRNAs, and other small interfering nucleic acid-based molecules known in the art. In one embodiment, vector-based RNAi modalities (e.g., shRNA or shRNA-mir expression constructs) are used to reduce expression of a gene (e.g., a HMG-CoA synthetase 2) in a cell. In some embodiments, therapeutic compositions of the invention comprise an isolated plasmid vector (e.g., any isolated plasmid vector known in the art or disclosed herein) that expresses a small interfering nucleic acid such as an shRNA. The isolated plasmid may comprise a tumor-specific, e.g., lung cancer-specific, promoter operably linked to a gene encoding the small interfering nucleic acid, e.g., an shRNA. In some cases, the isolated plasmid vector is packaged in a virus capable of infecting the individual. Exemplary viruses include adenovirus, retrovirus, lentivirus, adeno-associated virus, and others that are known in the art and disclosed herein. siRNA for HMGCS2 is commercially available from a variety of sources including Santa Cruz Biotechnology (cHMGCS siRNA (m): sc-44507) and Origene (HMGCS2 (ID 3158) Trilencer-27 Human siRNA).

A broad range of RNAi-based modalities could be employed to inhibit expression of a gene in a cell, such as siRNA-based oligonucleotides and/or altered siRNA-based oligonucleotides. Altered siRNA based oligonucleotides are those modified to alter potency, target affinity, safety profile and/or stability, for example, to render them resistant or partially resistant to intracellular degradation. Modifications, such as phosphorothioates, for example, can be made to oligonucleotides to increase resistance to nuclease degradation, binding affinity and/or uptake. In addition, hydrophobization and bioconjugation enhances siRNA delivery and targeting (De Paula et al., RNA. 13(4):431-56, 2007) and siRNAs with ribo-difluorotoluyl nucleotides maintain gene silencing activity (Xia et al., ASC Chem. Biol. 1(3):176-83, (2006)). siRNAs with amide-linked oligoribonucleosides have been generated that are more resistant to S1 nuclease degradation than unmodified siRNAs (Iwase R et al. 2006 Nucleic Acids Symp Ser 50: 175-176). In addition, modification of siRNAs at the 2′-sugar position and phosphodiester linkage confers improved serum stability without loss of efficacy (Choung et al., Biochem. Biophys. Res. Commun. 342(3):919-26, 2006). Other molecules that can be used to inhibit expression of a gene (e.g., a CSC-associated gene) include sense and antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat Med. 4(8):967-71, 1998). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; Jankowsky and Schwenzer Nucleic Acids Res. 24(3):423-9, 1996). Multitarget ribozymes (connected or shotgun) have been suggested as a means of improving efficiency of ribozymes for gene suppression (Ohkawa et al., Nucleic Acids Symp Ser. (29):121-2, 1993).

Triple helix approaches have also been investigated for sequence-specific gene suppression. Triple helix forming oligonucleotides have been found in some cases to bind in a sequence-specific manner (Postel et al., Proc. Natl. Acad. Sci. U.S.A. 88(18):8227-31, 1991; Duval-Valentin et al., Proc. Natl. Acad. Sci. U.S.A. 89(2):504-8, 1992; Hardenbol and Van Dyke Proc. Natl. Acad. Sci. U.S.A. 93(7):2811-6, 1996; Porumb et al., Cancer Res. 56(3):515-22, 1996). Similarly, peptide nucleic acids have been shown to inhibit gene expression (Hanvey et al., Antisense Res. Dev. 1(4):307-17, 1991; Knudsen and Nielson Nucleic Acids Res. 24(3):494-500, 1996; Taylor et al., Arch. Surg. 132(11):1177-83, 1997). Minor-groove binding polyamides can bind in a sequence-specific manner to DNA targets and hence may represent useful small molecules for suppression at the DNA level (Trauger et al., Chem. Biol. 3(5):369-77, 1996). In addition, suppression has been obtained by interference at the protein level using dominant negative mutant peptides and antibodies (Herskowitz Nature 329(6136):219-22, 1987; Rimsky et al., Nature 341(6241):453-6, 1989; Wright et al., Proc. Natl. Acad. Sci. U.S.A. 86(9):3199-203, 1989). The diverse array of suppression strategies that can be employed includes the use of DNA and/or RNA aptamers that can be selected to target a protein of interest (e.g., a HMG-CoA synthetase 2 polypeptide).

In one aspect, the invention provides methods for the treatment of cancer. “Cancer”, as used herein, refers to an uncontrolled growth of cells which interferes with the normal functioning of the bodily organs and systems. Cancers, including those cancers which migrate from their original location and seed vital organs, can eventually lead to the death of the subject through the functional deterioration of the affected organs. Cancers can be classified into a variety of categories including, carcinomas, sarcomas and hematopoietic cancers. Carcinomas are malignant cancers that arise from epithelial cells and include adenocarcinoma and squamous cell carcinoma.

The cancer of the invention is a cancer characterized by cells having increased NF-κB signaling. A preferred cancer of the invention is lung cancer, such as lung adenocarcinoma. Lung adenocarcinoma is a leading cause of cancer death worldwide. A cancer “characterized by cells having increased NF-κB signaling”, is a cancer comprising cancer cells that have increased NF-κ signaling. The cancer can consist exclusively of cells that have increased NF-κB signaling or the cancer may include a subpopulation of cells that have increased NF-κB signaling. Cells having “increased NF-κB signaling” are cells that have increased NF-κB signaling compared to wild-type (“normal” or “non-cancerous”) cells. Increased NF-κB signaling can be caused, for instance, by activating mutations in one or more critical molecules in the NF-κB pathway, or by overexpression of one or more molecules in the NF-κB pathway. Cells with increased NF-κB signaling can be identified, for example, by assaying the levels and/or activation status of NF-κB proteins or RNA in the pathway. Assays that can detect the level or activation status of proteins or nucleic acids are known in the art and include western blots, northern blots, and protein array analysis. Increased intracellular signaling, as compared to wild-type cells, includes 1% or more, 5% or more, 10% or more, 20% or more, 50% or more, 100% or more, 5× or more, 10× or more, 100× or more, 1000× or more intracellular signaling.

The cancer characterized by cells having increased NF-κB signaling is associated with genetic mutations in some embodiments of the invention. For instance cancer cells having Kras and p53 mutations are particularly susceptible to the therapy.

The amount of NF-κB signaling or marker gene expression level in a cancerous tissue is preferably measured in a sample from a patient to be treated. In some embodiments, the amount is measured in vivo in a subject using one or more methods described herein or known in the art. In other embodiments, the amount is measured in a sample obtained from a subject suspected of having cancer or a patient diagnosed as having cancer. The sample can be a solid tissue biopsy or a biological fluid sample. The sample can contain essentially cancer cells. Alternatively, the sample can contain a mixture of cancer cells and non-cancer cells. The amount of NF-κB signaling or marker gene expression can be obtained directly or extrapolated using appropriate controls and/or standards. According to the invention, the amount of NF-κB signaling or marker gene expression can be measured as a protein amount, a nucleic acid amount (e.g. an mRNA amount) or an activity amount. The tissue sample may include any body tissue or fluid that is suspected of harboring the cancer cells. For example, the cancer cells are commonly found in or around a tumor mass for solid tumors.

As used herein, a subject is a human, non-human primate, cow, horse, pig, sheep, goat, dog, cat, or rodent. In all embodiments human subjects are preferred. In aspects of the invention pertaining to predictive therapy in cancers, the subject is a human either suspected of having the cancer, or having been diagnosed with cancer. Methods for identifying subjects suspected of having cancer may include physical examination, subject's family medical history, subject's medical history, biopsy, or a number of imaging technologies such as ultrasonography, computed tomography, magnetic resonance imaging, magnetic resonance spectroscopy, or positron emission tomography. Diagnostic methods for cancer and the clinical delineation of cancer diagnoses are well known to those of skill in the medical arts.

As used herein, a tissue sample is tissue obtained from a tissue biopsy, a surgically resected tumor, or any other tumor cell mass removed from the body using methods well known to those of ordinary skill in the related medical arts. The phrase “suspected of being cancerous” as used herein means a cancer tissue sample believed by one of ordinary skill in the medical arts to contain cancerous cells. Methods for obtaining the sample from a biopsy include gross apportioning of a mass, microdissection, laser-based microdissection, or other art-known cell-separation methods.

Because of the variability of the cell types in diseased-tissue biopsy material, and the variability in sensitivity of the predictive methods used, the sample size required for analysis may range from 1, 10, 50, 100, 200, 300, 500, 1000, 5000, 10,000, to 50,000 or more cells. The appropriate sample size may be determined based on the cellular composition and condition of the biopsy and the standard preparative steps for this determination and subsequent isolation of the nucleic acid for use in the invention are well known to one of ordinary skill in the art. An example of this, although not intended to be limiting, is that in some instances a sample from the biopsy may be sufficient for assessment of RNA expression without amplification, but in other instances the lack of suitable cells in a small biopsy region may require use of RNA conversion and/or amplification methods or other methods to enhance resolution of the nucleic acid molecules or proteins. Such methods, which allow use of limited biopsy materials, are well known to those of ordinary skill in the art and include, but are not limited to: direct RNA amplification, reverse transcription of RNA to cDNA, amplification of cDNA, or the generation of radio-labeled nucleic acids or proteins.

According to some embodiments of the invention, a marker reference or threshold amount is an amount that is known (or is shown) to be an amount above which a cell (e.g. a cancer cell) is not responsive to NF-κB treatment. In other embodiments, a marker reference or threshold amount is an amount that is known (or is shown) to be an amount below which a cell (e.g. a cancer cell) is responsive or susceptible to NF-κB treatment.

The actual numbers in the particular determination of threshold values may vary for different tumors or under different circumstances, such as the conditions of the assay to determine expression. However, the skilled artisan would be able to identify the correct threshold values based on the circumstances. For example threshold values could easily be generated using normal non-cancerous tissue under similar circumstances. In each instance, the comparison of the expression levels of markers to a reference value is useful in determining the relative levels of marker in the test tumor cells.

The reference sample can be any of a variety of biological samples against which a diagnostic assessment may be made. Examples of reference samples include biological samples from control populations or control samples. Reference samples may be generated through manufacture to be supplied for testing in parallel with the test samples, e.g., reference sample may be supplied in diagnostic kits. Appropriate reference samples will be apparent to the skilled artisan.

In other embodiments, the expression level of the marker gene or protein in the test sample may be determined based on a direct comparison to a reference level in absolute values. For instance, at least 10%, at least 20%, at least 50%, at least 100%, at least 200%, at least 500%, at least 1000% or more higher than the expression level of the gene or protein in the reference sample. In other embodiments, the expression level of the marker in the test sample is at least 10%, at least 20%, at least 50%, at least 100%, at least 200%, at least 500%, at least 1000% or more lower than the expression level of the gene or protein in the reference sample. The magnitude of difference between an expression level and an appropriate reference level may vary. For example, a significant difference that indicates a particular therapeutic regimen is called for may be detected when the expression level of the marker gene in a clinical sample is at least 1%, at least 5%, at least 10%, at least 25%, at least 50%, at least 100%, at least 250%, at least 500%, or at least 1000% higher, or lower, than an appropriate reference level of that gene. Similarly, a significant difference may be detected when the expression level of a marker gene in a clinical sample is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the appropriate reference level of that gene. Significant differences may be identified by using an appropriate statistical test. Tests for statistical significance are well known in the art and are exemplified in Applied Statistics for Engineers and Scientists by Petruccelli, Chen and Nandram 1999 Reprint Ed. It is to be understood that a plurality of expression levels may be compared with plurality of appropriate reference levels, e.g., on a gene-by-gene basis in order to assess the status of the individual. In such cases, Multivariate Tests, e.g., Hotelling's T² test, may be used to evaluate the significance of observed differences. Such multivariate tests are well known in the art and are exemplified in Applied Multivariate Statistical Analysis by Richard Arnold Johnson and Dean W. Wichern Prentice Hall; 4^{th} edition (Jul. 13, 1998).

The marker levels may be measured using any of a number of techniques available to the person of ordinary skill in the art for protein or nucleic acid, e.g., direct physical measurements (e.g., mass spectrometry), binding assays (e.g., immunoassays, agglutination assays, and immunochromatographic assays), Polymerase Chain Reaction (PCR) technology, branched oligonucleotide technology, Northern blot technology, oligonucleotide hybridization technology, and in situ hybridization technology. The method may also comprise measuring a signal that results from a chemical reaction, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques may detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques may be used that do not require the use of labels, e.g., techniques based on measuring mass (e.g., surface acoustic wave measurements), refractive index (e.g., surface plasmon resonance measurements), or the inherent luminescence of an analyte.

The methods may involve the steps of isolating nucleic acids from the sample and/or an amplification step. Typically, a nucleic acid comprising a sequence of interest can be obtained from a biological sample, more particularly from a sample comprising DNA (e.g. gDNA or cDNA) or RNA (e.g. mRNA). Release, concentration and isolation of the nucleic acids from the sample can be done by any method known in the art. Various commercial kits are available such as the High pure PCR Template Preparation Kit (Roche Diagnostics, Basel, Switzerland) or the DNA purification kits (PureGene, Gentra, Minneapolis, US). Other, well-known procedures for the isolation of DNA or RNA from a biological sample are also available (Sambrook et al., Cold Spring Harbor Laboratory Press 1989, Cold Spring Harbor, N.Y., USA; Ausubel et al., Current Protocols in Molecular Biology 2003, John Wiley & Sons).

When the quantity of the nucleic acid is low or insufficient for the assessment, the nucleic acid of interest may be amplified. Such amplification procedures can be accomplished by those methods known in the art, including, for example, the polymerase chain reaction (PCR), ligase chain reaction (LCR), nucleic acid sequence-based amplification (NASBA), strand displacement amplification, rolling circle amplification, T7-polymerase amplification, and reverse transcription polymerase reaction (RT-PCR).

Polymerase chain reaction (PCR) technology is practiced routinely by those having ordinary skill in the art and its uses in diagnostics are well known and accepted. Methods for practicing PCR technology are disclosed in “PCR Protocols: A Guide to Methods and Applications”, Innis, M. A., et al. Eds. Academic Press, Inc. San Diego, Calif. (1990) which is incorporated herein by reference. Applications of PCR technology are disclosed in “Polymerase Chain Reaction” Erlich, H. A., et al., Eds. Cold Spring Harbor Press, Cold Spring Harbor, N.Y. (1989) which is incorporated herein by reference. U.S. Pat. No. 4,683,202, U.S. Pat. No. 4,683,195, U.S. Pat. No. 4,965,188 and U.S. Pat. No. 5,075,216, which are each incorporated herein by reference describe methods of performing PCR. PCR technology allows for the rapid generation of multiple copies of DNA sequences by providing 5′ and 3′ primers that hybridize to sequences present in an RNA or DNA molecule, and further providing free nucleotides and an enzyme which fills in the complementary bases to the nucleotide sequence between the primers with the free nucleotides to produce a complementary strand of DNA.

PCR primers can be designed routinely by those having ordinary skill in the art using sequence information. The mRNA or cDNA is combined with the primers, free nucleotides and enzyme following standard PCR protocols. The mixture undergoes a series of temperature changes. If the test gene transcript or cDNA generated therefrom is present, that is, if both primers hybridize to sequences on the same molecule, the molecule comprising the primers and the intervening complementary sequences will be exponentially amplified. The amplified DNA can be easily detected by a variety of well-known means. If no gene transcript or cDNA generated therefrom is present, no PCR product will be exponentially amplified.

PCR product may be detected by several well-known means. One method for detecting the presence of amplified DNA is to separate the PCR reaction material by gel electrophoresis and stain the gel with ethidium bromide in order to visual the amplified DNA if present. A size standard of the expected size of the amplified DNA is preferably run on the gel as a control.

In some instances, such as when unusually small amounts of RNA are recovered and only small amounts of cDNA are generated therefrom, it is desirable to perform a PCR reaction on the first PCR reaction product. The second PCR can be performed to make multiple copies of DNA sequences of the first amplified DNA. A nested set of primers are used in the second PCR reaction. The nested set of primers hybridize to sequences downstream of the 5′ primer and upstream of the 3′ primer used in the first reaction.

Branched chain oligonucleotide hybridization may be performed as described in U.S. Pat. No. 5,597,909, U.S. Pat. No. 5,437,977 and U.S. Pat. No. 5,430,138, which are each incorporated herein by reference. Northern blot analysis methods are well known by those having ordinary skill in the art and are described in Sambrook, J. et al., (1989) Molecular Cloning: A Laboratory Manual, Cold Spring Harbor Laboratory Press, Cold Spring Harbor, N.Y. Additionally, mRNA extraction, electrophoretic separation of the mRNA, blotting, probe preparation and hybridization are all well-known techniques that can be routinely performed using readily available starting material.

Hybridization methods for nucleic acids are well known to those of ordinary skill in the art (see, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York). The nucleic acid molecules hybridize under stringent conditions to nucleic acid markers expressed in cancer cells. The nucleic acid markers disclosed herein are known genes and fragments thereof. Targets are nucleic acids selected from the group, including but not limited to: DNA, genomic DNA, cDNA, RNA, mRNA and may be natural or synthetic.

Binding assays for measuring marker levels may use solid phase or homogenous formats. Suitable assay methods include sandwich or competitive binding assays. Examples of sandwich immunoassays are described in U.S. Pat. No. 4,168,146 to Grubb et al. and U.S. Pat. No. 4,366,241 to Tom et al., both of which are incorporated herein by reference. Examples of competitive immunoassays include those disclosed in U.S. Pat. No. 4,235,601 to Deutsch et al., U.S. Pat. No. 4,442,204 to Liotta, and U.S. Pat. No. 5,208,535 to Buechler et al., all of which are incorporated herein by reference.

Multiple markers may be measured using a multiplexed assay format, e.g., multiplexing through the use of binding reagent arrays, multiplexing using spectral discrimination of labels, multiplexing by flow cytometric analysis of binding assays carried out on particles (e.g., using the Luminex system). Thus, in some embodiments the invention encompasses arrays of nucleic acid or peptide based detection reagents for assaying 10 or more, 20 or more, 30 or more, 40 or more, 50 or more, or 100 or more markers from Table 1.

Detection of a protein in a test sample involves routine methods. The skilled artisan can detect the presence or absence of a protein using well known methods. One such method is an immunoassay. In general, immunoassays involve the binding of proteins in a sample to a solid phase support such as a plastic surface. Detectable antibodies are then added which selectively bind to the protein of interest. Detection of the antibody indicates the presence of the protein. The detectable antibody may be a labeled or an unlabeled antibody. Unlabeled antibody may be detected using a second, labeled antibody that specifically binds to the first antibody or a second, unlabeled antibody which can be detected using labeled protein A, a protein that complexes with antibodies. Various immunoassay procedures are described in Immunoassays for the 80's, A. Voller et al., Eds., University Park, 1981, which is incorporated herein by reference.

Simple immunoassays such as a dot blot and a Western blot involve the use of a solid phase support which is contacted with a test sample. Any proteins present in the test sample bind the solid phase support and can be detected by a specific, detectable antibody preparation. The intensity of the signal can be measured to obtain a quantitative readout. Other more complex immunoassays include forward assays for the detection of a protein in which a first anti-protein antibody bound to a solid phase support is contacted with the test sample. After a suitable incubation period, the solid phase support is washed to remove unbound protein. A second, distinct anti-protein antibody is then added which is specific for a portion of the specific protein not recognized by the first antibody. The second antibody is preferably detectable. After a second incubation period to permit the detectable antibody to complex with the specific protein bound to the solid phase support through the first antibody, the solid phase support is washed a second time to remove the unbound detectable antibody. Alternatively, in a forward sandwich assay a third detectable antibody, which binds the second antibody is added to the system. Other types of immunometric assays include simultaneous and reverse assays. A simultaneous assay involves a single incubation step wherein the first antibody bound to the solid phase support, the second, detectable antibody and the test sample are added at the same time. After the incubation is completed, the solid phase support is washed to remove unbound proteins. The presence of detectable antibody associated with the solid support is then determined as it would be in a conventional assays. A reverse assay involves the stepwise addition of a solution of detectable antibody to the test sample followed by an incubation period and the addition of antibody bound to a solid phase support after an additional incubation period. The solid phase support is washed in conventional fashion to remove unbound protein/antibody complexes and unreacted detectable antibody.

A number of methods are well known for the detection of antibodies. For instance, antibodies can be detectably labeled by linking the antibodies to an enzyme and subsequently using the antibodies in an enzyme immunoassay (EIA) or enzyme-linked immunosorbent assay (ELISA), such as a capture ELISA. The enzyme, when subsequently exposed to its substrate, reacts with the substrate and generates a chemical moiety which can be detected, for example, by spectrophotometric, fluorometric or visual means. Enzymes which can be used to detectably label antibodies include, but are not limited to malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

A detectable label is a moiety, the presence of which can be ascertained directly or indirectly. Generally, detection of the label involves an emission of energy by the label. The label can be detected directly by its ability to emit and/or absorb photons or other atomic particles of a particular wavelength (e.g., radioactivity, luminescence, optical or electron density, etc.). A label can be detected indirectly by its ability to bind, recruit and, in some cases, cleave another moiety which itself may emit or absorb light of a particular wavelength (e.g., epitope tag such as the FLAG epitope, enzyme tag such as horseradish peroxidase, etc.). An example of indirect detection is the use of a first enzyme label which cleaves a substrate into visible products. The label may be of a chemical, peptide or nucleic acid molecule nature although it is not so limited. Other detectable labels include radioactive isotopes such as P³² or H³, luminescent markers such as fluorochromes, optical or electron density markers, etc., or epitope tags such as the FLAG epitope or the HA epitope, biotin, avidin, and enzyme tags such as horseradish peroxidase, β-galactosidase, etc. The label may be bound to a peptide during or following its synthesis. There are many different labels and methods of labeling known to those of ordinary skill in the art. Examples of the types of labels that can be used in the present invention include enzymes, radioisotopes, fluorescent compounds, colloidal metals, chemiluminescent compounds, and bioluminescent compounds. Those of ordinary skill in the art will know of other suitable labels for the peptides described herein, or will be able to ascertain such, using routine experimentation. Furthermore, the coupling or conjugation of these labels to the peptides of the invention can be performed using standard techniques common to those of ordinary skill in the art.

Another labeling technique which may result in greater sensitivity consists of coupling the molecules described herein to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten antibodies.

Conjugation of the peptides including antibodies or fragments thereof to a detectable label facilitates, among other things, the use of such agents in diagnostic assays. Another category of detectable labels includes diagnostic and imaging labels (generally referred to as in vivo detectable labels) such as for example magnetic resonance imaging (MRI): Gd(DOTA); for nuclear medicine: ²⁰¹Tl, gamma-emitting radionuclide 99mTc; for positron-emission tomography (PET): positron-emitting isotopes, (18)F-fluorodeoxyglucose ((18)FDG), (18)F-fluoride, copper-64, gadodiamide, and radioisotopes of Pb(II) such as 203Pb; 111In.

As used herein, “conjugated” means two entities stably bound to one another by any physiochemical means. It is important that the nature of the attachment is such that it does not impair substantially the effectiveness of either entity. Keeping these parameters in mind, any covalent or non-covalent linkage known to those of ordinary skill in the art may be employed. In some embodiments, covalent linkage is preferred. Noncovalent conjugation includes hydrophobic interactions, ionic interactions, high affinity interactions such as biotin-avidin and biotin-streptavidin complexation and other affinity interactions. Such means and methods of attachment are well known to those of ordinary skill in the art.

The therapeutic compounds described herein can be administered in combination with other therapeutic agents and such administration may be simultaneous or sequential. When the other therapeutic agents are administered simultaneously they can be administered in the same or separate formulations, but are administered at the same time. The administration of the other therapeutic agent, including chemotherapeutics can also be temporally separated, meaning that the therapeutic agents are administered at a different time, either before or after, the administration of the therapeutics described herein. The separation in time between the administration of these compounds may be a matter of minutes or it may be longer.

Thus, in some instances, the invention also involves administering another cancer treatment (e.g., radiation therapy, chemotherapy or surgery) to a subject. Examples of conventional cancer therapies include treatment of the cancer with agents such as All-trans retinoic acid, Actinomycin D, Adriamycin, anastrozole, Azacitidine, Azathioprine, Alkeran, Ara-C, Arsenic Trioxide (Trisenox), BiCNU Bleomycin, Busulfan, CCNU, Carboplatin, Capecitabine, Cisplatin, Chlorambucil, Cyclophosphamide, Cytarabine, Cytoxan, DTIC, Daunorubicin, Docetaxel, Doxifluridine, Doxorubicin, 5-flurouracil, Epirubicin, Epothilone, Etoposide, exemestane, Erlotinib, Fludarabine, Fluorouracil, Gemcitabine, Hydroxyurea, Herceptin, Hydrea, Ifosfamide, Irinotecan, Idarubicin, Imatinib, letrozole, Lapatinib, Leustatin, 6-MP, Mithramycin, Mitomycin, Mitoxantrone, Mechlorethamine, megestrol, Mercaptopurine, Methotrexate, Mitoxantrone, Navelbine, Nitrogen Mustard, Oxaliplatin, Paclitaxel, pamidronate disodium, Pemetrexed, Rituxan, 6-TG, Taxol, Topotecan, tamoxifen, taxotere, Teniposide, Tioguanine, toremifene, trimetrexate, trastuzumab, Valrubicin, Vinblastine, Vincristine, Vindesine, Vinorelbine, Velban, VP-16, and Xeloda.

Other therapeutics for cancer involve antibodies or other binding proteins conjugated to a cytotoxic agents. The conjugates include an antibody conjugated to a cytotoxic agent such as a chemotherapeutic agent, toxin (e.g. an enzymatically active toxin of bacterial, fungal, plant or animal origin, or fragments thereof, or a small molecule toxin), or a radioactive isotope (i.e., a radioconjugate). Other antitumor agents that can be conjugated to the antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296). Enzymatically active toxins and fragments thereof which can be used in the conjugates include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes.

For selective destruction of the cell, the antibody may comprise a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for detection, it may comprise a radioactive atom for scintigraphic studies, for example tc⁹⁹m or I¹²³, or a spin label for nuclear magnetic resonance (NMR) imaging (also known as magnetic resonance imaging, mri), such as iodine-123, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

When used in combination with the therapies of the invention the dosages of known therapies may be reduced in some instances, to avoid side effects.

Cancer therapies and their dosages, routes of administration and recommended usage are known in the art and have been described in such literature as the Physician's Desk Reference (56^{th} ed., 2002). In some embodiments, the therapeutic compounds of the invention are formulated into a pharmaceutical composition that further comprises one or more additional anticancer agents.

The active agents of the invention are administered to the subject in an effective amount for treating the subject. An “effective amount”, for instance, is an amount necessary or sufficient to realize a desired biologic effect. For instance an effective amount is that amount sufficient to prevent or inhibit cancer cell growth or proliferation or alternatively an amount sufficient to induce apoptosis of a cancer cell or induce tumor regression.

The effective amount of a compound of the invention in the treatment of a subject may vary depending upon the specific compound used, the mode of delivery of the compound, and whether it is used alone or in combination. The effective amount for any particular application can also vary depending on such factors as the type and/or degree of cancer in a subject, the particular compound being administered for treatment, the size of the subject, or the severity of the disorder. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity in and of itself and yet is entirely effective to treat the particular subject.

Toxicity and efficacy of the protocols of the present invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays, animal studies and human studies can be used in formulating a range of dosage of the prophylactic and/or therapeutic agents for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

As used herein, a “level” refers to a value indicative of the amount or occurrence of a molecule, e.g., a protein, a nucleic acid, e.g., RNA. A level may be an absolute value, e.g., a quantity of a molecule in a sample, or a relative value, e.g., a quantity of a molecule in a sample relative to the quantity of the molecule in a reference sample (control sample). The level may also be a binary value indicating the presence or absence of a molecule. For example, a molecule may be identified as being present in a sample when a measurement of the quantity of the molecule in the sample, e.g., a fluorescence measurement from a PCR reaction or microarray, exceeds a background value. Similarly, a molecule may be identified as being absent from a sample (or undetectable in a sample) when a measurement of the quantity of the molecule in the sample is at or below background value.

As used herein, the term treat, treated, or treating when used with respect to a disorder refers to a prophylactic treatment which increases the resistance of a subject to development of the disease or, in other words, decreases the likelihood that the subject will develop the disease as well as a treatment after the subject has developed the disease in order to fight the disease, prevent the disease from becoming worse, or slow the progression of the disease compared to in the absence of the therapy.

Multiple doses of the molecules of the invention are also contemplated. In some instances, when the molecules of the invention are administered with another therapeutic, for instance, a chemotherapeutic agent a sub-therapeutic dosage of either or both of the molecules may be used. A “sub-therapeutic dose” as used herein refers to a dosage which is less than that dosage which would produce a therapeutic result in the subject if administered in the absence of the other agent.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards. The compounds are generally suitable for administration to humans. This term requires that a compound or composition be nontoxic and sufficiently pure so that no further manipulation of the compound or composition is needed prior to administration to humans.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated. The compounds may be sterile or non-sterile.

The agent may comprise different types of carriers depending on whether it is to be administered in solid, liquid or aerosol form, and whether it need to be sterile for such routes of administration as injection. The present invention can be administered intravenously, intradermally, intraarterially, intralesionally, intratumorally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences (1990), incorporated herein by reference). In a particular embodiment, intraperitoneal injection is contemplated.

In any case, the composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The agent may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The compounds of the invention may be administered directly to a tissue. Direct tissue administration may be achieved by direct injection. The compounds may be administered once, or alternatively they may be administered in a plurality of administrations. If administered multiple times, the compounds may be administered via different routes. For example, the first (or the first few) administrations may be made directly into the affected tissue while later administrations may be systemic.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients. In general, a pharmaceutical composition comprises the compound of the invention and a pharmaceutically-acceptable carrier. Pharmaceutically-acceptable carriers for nucleic acids, small molecules, peptides, monoclonal antibodies, and antibody fragments are well-known to those of ordinary skill in the art. As used herein, a pharmaceutically-acceptable carrier means a non-toxic material that does not interfere with the effectiveness of the biological activity of the active ingredients.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compounds of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids, such as a syrup, an elixir or an emulsion.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the active agent (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In one embodiment, a kit comprises a reagent for detecting a marker shown in Table 1 or for detecting NF-κB signaling. The kit may further comprise assay diluents, standards, controls and/or detectable labels. The assay diluents, standards and/or controls may be optimized for a particular sample matrix. Reagents include, for instance, antibodies, nucleic acids, labeled secondary agents, or in the alternative, if the primary reagent is labeled, enzymatic or agent binding reagents which are capable of reacting with the labeled reagent. One skilled in the art will readily recognize that reagents of the present invention can be readily incorporated into one of the established kit formats which are well known in the art.

As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention in connection with treatment or characterization of a cancer.

“Instructions” can define a component of promotion, and typically involve written instructions on or associated with packaging of compositions of the invention. Instructions also can include any oral or electronic instructions provided in any manner.

Thus the agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended therapeutic application and the proper administration of these agents. In certain embodiments agents in a kit may be in a pharmaceutical formulation and dosage suitable for a particular application and for a method of administration of the agents.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for human administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Methods

Mice and Drug Treatment:

The Massachusetts Institute of Technology (Cambridge, Mass.) Institutional Animal Care and Use Committee approved all animal studies and procedures. To initiate lung tumors, cohorts of K or KP mice of 129svJae background were infected with 2.5×10⁷ plaque-forming units of Adeno-Cre (University of Iowa) by intranasal inhalation, as described previously (2, 28). Mice were given bortezomib (LC Labs) in PBS [0.5% dimethylsulfoxide (DMSO)] at 1 mg/kg body weight i.v., as indicated. Bay-117082 (CalBiochem) was dissolved in DMSO, diluted in PBS as a fine suspension, and injected at 10 mg/kg body weight i.p., as indicated.

Immunohistochemistry:

Mice were sacrificed by carbon dioxide asphyxiation. Lungs were inflated with 4% formalin (neutral buffered formalin), fixed overnight, and transferred to 70% ethanol. Lung lobes were embedded in paraffin and sectioned at 4 μm and stained with H&E for tumor pathologic study. For staining with anti-CC3 antibodies (#9661; Cell Signaling), lung tumor sections were dewaxed, rehydrated, and subjected to high-temperature antigen retrieval—10 minutes of boiling in a pressure cooker in 0.01 M citrate buffer, pH 6.0. Slides were stained overnight at 4° in 1:100 primary antibody. A goat anti-rabbit horseradish peroxidase-conjugated secondary antibody (Vector Laboratories) was used at 1:200 dilution, incubated for 1 hour at room temperature, followed by diaminobenzidine staining (Vector Laboratories). The number of positive cells per tumor area was quantified using Bioquant software from >10 tumors in ≧3 mice per group (2).

MicroCT and Bioluminescence Imaging:

At indicated time points, mice were scanned for 15 min under isoflurane anesthesia, using a small animal eXplore Locus microCT (GE Healthcare) at 45-μm resolution, 80 kV, with 450-mA current (47). Images were acquired and processed using GE eXplore software. Bioluminescence imaging was performed as previously described (48). Mice were imaged for 60 seconds, and signals in the lung were quantified using Xenogen software.

Immunoblotting and Immunofluorescence:

Cell pellets were lysed in Laemmli buffer. Equal amounts of protein (16 μg) were separated on 10% SDS-polyacrylamide gels and transferred to polyvinylidene difluoride membranes. Blots were probed with antibodies (1:1000 dilution) against p65 (c-20), NEMO (FL-419), Parp (46D11), c-Rel (C), p100/p52 (#4882; Cell Signaling), or CC3 (#9661; Cell Signaling). Nuclear-cytoplasmic fractionations and NF-κB p65 DNA-binding activity assay were as described recently (30). For the NF-κB subunit DNA-binding activity assay, 5 μg of nuclear extracts was used to determine NF-κB DNA-binding activity with anti-p65, p52, p50, RelB, or C-Rel primary antibodies in an ELISA-based assay, according to the manufacturer's instructions (TransAM; Active Motif). Immunofluorescence was performed as recently described (49). Antibodies are as follows: p65 (c-20, 1:100), p100/p52 (#4882, 1:100; Cell Signaling), and goat anti-rabbit Alexa 488 (1:1000; Invitrogen). Cells were counterstained with 4,6-diamidino-2-phenylindole (DAPI; Sigma-Aldrich) and mounted in Vectashield antifade mountant (Vector Laboratories).

Cell Viability Assay:

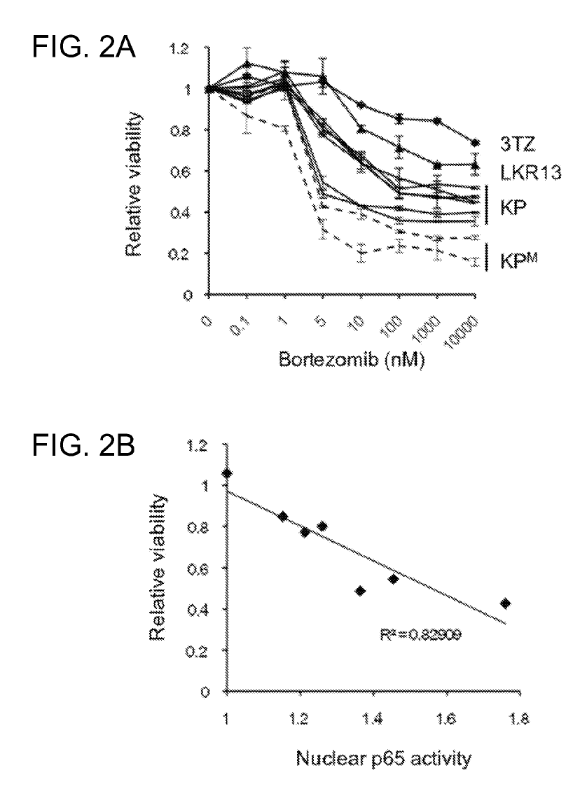

Cell culture conditions were as described recently (30). Cells were split into 96-well plates (5,000 cells per well). After 24 hours, cells were treated with bortezomib, and 24 hours later cell viability was measured by the Cell Titer Aqueous Kit (Promega) in triplicates. Vehicle control-treated cell values were set to 1 (100% viability). For FIG. 2A and FIG. 12A, the data are representative of 2 independent experiments.

Gene Expression Analysis:

RNA was purified using TRIzol (Invitrogen), according to the manufacturer's instructions. One microgram of RNA was reverse transcribed using the High-Capacity cDNA Reverse Transcription Kit (Applied Biosystems). Real-time PCR (quantitative PCR) amplification was performed using Taqman probes (Applied Biosystems). Data were normalized to the Gapdh (mouse) or GAPDH (human) levels.

Statistics: P values were determined by Student t tests.

### Example 1

### Bortezomib Inhibits NF-κB Signaling and Induces Apoptosis in Lung Adenocarcinoma Cells

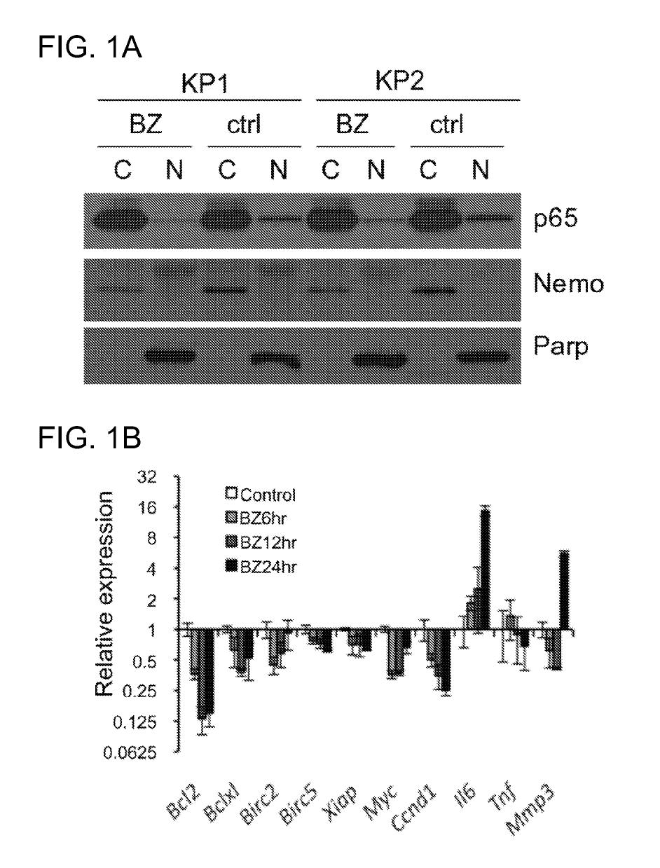

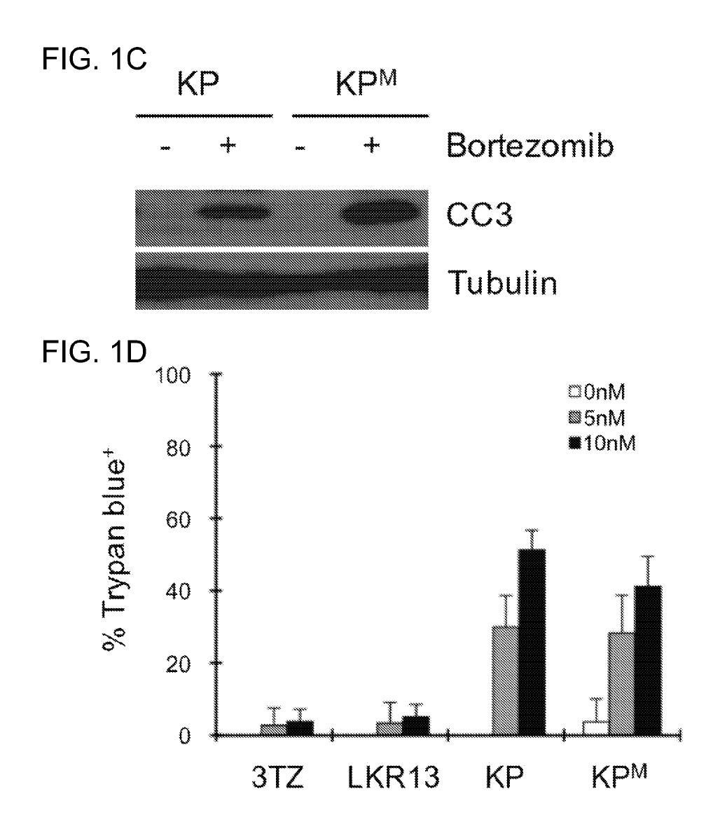

Bortezomib has been shown to inhibit NF-κB by suppressing proteasome-mediated IκB degradation (19, 33). We tested whether bortezomib would inhibit NF-κB signaling in lung adenocarcinoma cells. We have generated a panel of genetically defined mouse lung adenocarcinoma cell lines (hereafter termed “KP”) from tumors carrying a Cre-activatable KrasG12D allele (KrasLSL-G12D/WT; LSL denotes Lox-stop-Lox) and a conditional loss-of-function p53 allele (p53flox/flox). As shown in FIG. 1A, bortezomib-treated cells, compared with vehicle control-treated cells, showed reduced nuclear accumulation of the NF-κB transcription factor subunit p65 (also known as RelA). Because nuclear p65 is required for NF-κB activity, these data suggest that bortezomib is able to inhibit the NF-κB pathway in mouse lung adenocarcinoma cells.

To explore the molecular and cellular effects of NF-κB inhibition in KP cells, we examined the expression levels of known NF-κB target genes by real-time PCR after a time course of bortezomib treatment (FIG. 1B). Of note, NF-κB-regulated antiapoptosis genes, such as Bcl2, Bclx1, Birc2 (cIAP1), and Birc5 (Survivin), were consistently downregulated at all time points tested, demonstrating the efficiency of NF-κB inhibition. Furthermore, proliferation related NF-κB target genes, including c-Myc and Cyclin D1, also reduced expression following bortezomib treatment (FIG. 1B). Contrary to our expectations, the expression of 3 NF-κB targets that regulate inflammation, I16, Tnf, and Mmp3, was not reduced after treatment, and the I16 mRNA level was actually increased in cells treated with bortezomib. Induction of the inflammatory genes may be due to secondary effects of proteasome inhibition in these cells. Future experiments will examine the importance of bortezomib's proinflammatory effects in tumor cells.

As the NF-κB pathway is known to inhibit apoptosis through its regulation of antiapoptotic genes, we next addressed the cytotoxicity of bortezomib in vitro (6). Consistent with decreased levels of Bcl2 and other antiapoptotic genes in bortezomib-treated cells (FIG. 1B), we observed increased cleaved caspase 3 (CC3) in KP, as well as in cells harboring the KrasG12D mutation and a point mutation (R172H) in p53 (KP^{M}; FIG. 1C). We also performed Trypan blue counting and confirmed that bortezomib treatment caused cell death in a dose-dependent manner in KP and KP^{M} cells. Of interest, LKR13 cells, which express mutant Kras but retain wild-type p53 expression and 3TZ fibroblasts (30), did not show substantial cell death under the assayed conditions.

We also tested bortezomib in 2 human NSCLC cell lines that contain mutations in KRAS and loss of function in p53 (FIG. 8). Consistent with the mouse data, the NF-κB target genes MYC, BCL2, and XIAP were downregulated in bortezomib-treated human cells.

### Example 2

### Bortezomib Sensitivity Correlates with Basal NF-κB Activity in KP Lung Adenocarcinoma Cells

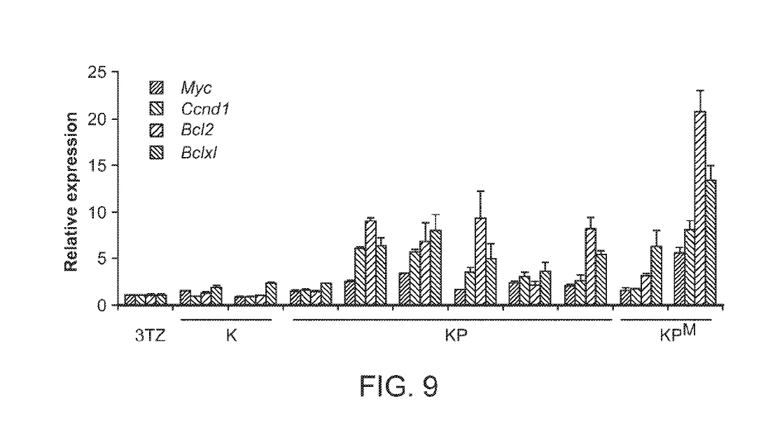

To investigate the dose-response profile of bortezomib, we treated a panel of KP and KP^{M} cells with increasing doses of bortezomib for 24 hours and monitored cell viability. As shown in FIG. 2A, KP and KP^{M} cell lines showed higher sensitivity to the drug than did control 3TZ and Kras-only LKR13 cells. We previously showed that in KP cell lines, NF-κB p65 DNA-binding activity was consistently higher than in 3TZ and LKR13 cells (30). By measuring NF-κB target gene expression, we observed that KP and KP^{M} cells also have higher NF-κB target gene expression than do 3TZ or LKR13 cells (FIG. 9). To examine whether the level of NF-κB activity might be a biomarker to predict bortezomib response, we measured NF-κB activity in KP cell lines, using ELISA (30), and quantified cell viability at 5 nM bortezomib treatment. In the cell lines assayed, NF-κB activity was positively correlated with bortezomib-induced cytotoxicity (FIG. 2B), with cell lines that had high NF-κB activity levels exhibiting more sensitivity to the drug than cells with lower NF-κB levels. Thus, we conclude that lung adenocarcinoma cells with high NF-κB signaling are dependent on continuous activation of this pathway. These data are consistent with studies showing that multiple myelomas with high NF-κB activity are more sensitive to bortezomib (22) and IKK inhibitors (13).

### Example 3

### Bortezomib Leads to Lung Tumor Regression in KP Mice

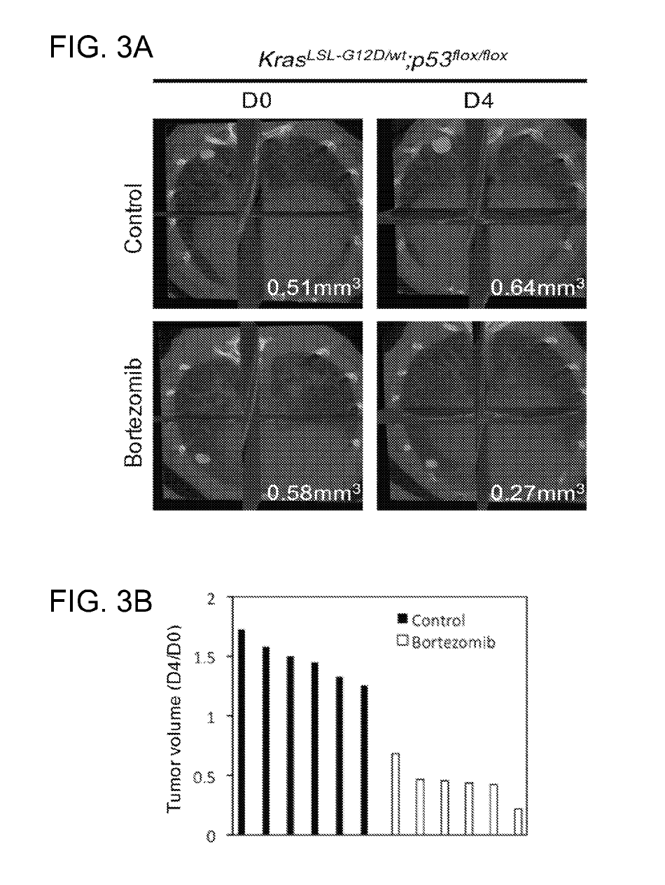

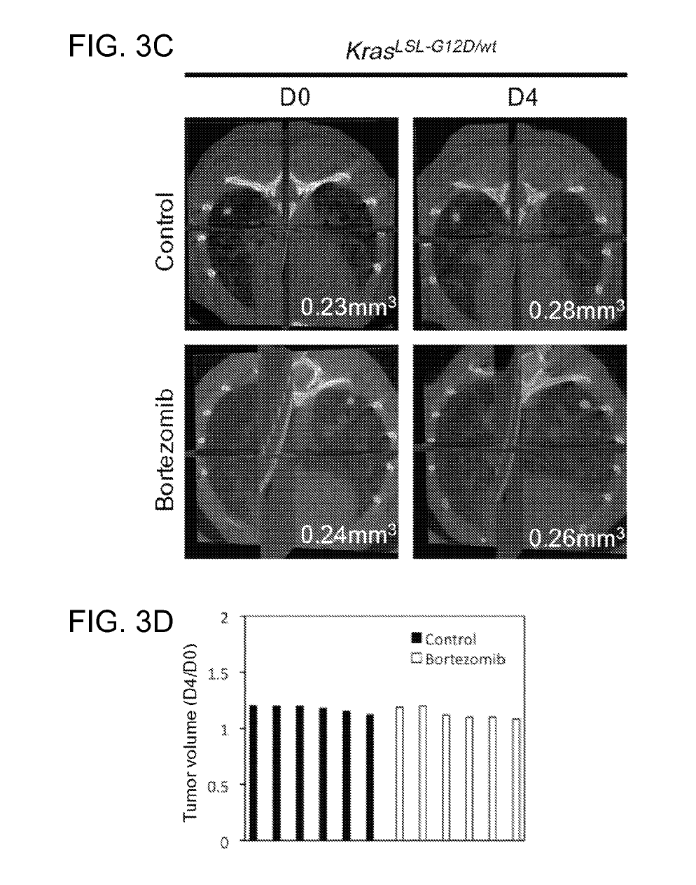

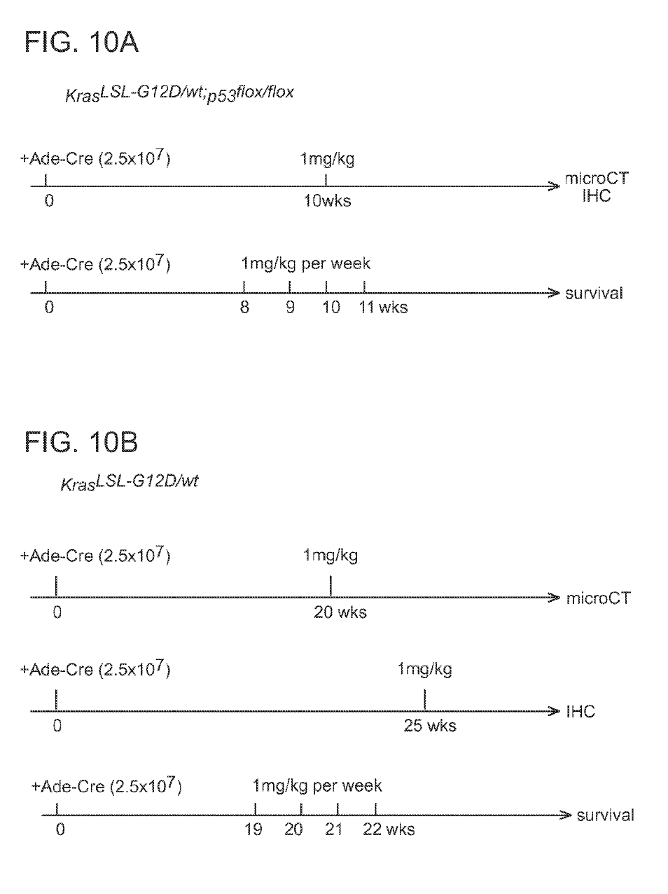

Our cell-based studies showed that bortezomib induced apoptosis in murine lung adenocarcinoma cell lines grown in culture (FIG. 1C). To understand the relevance of these findings in vivo, we examined bortezomib-mediated NF-κB inhibition in both KP and K models of lung cancer. Previous data have shown that KP lung tumors have higher NF-κB signaling than do those from the K model (30). To test the functional requirement for NF-κB in KP tumors, we infected 6- to 8-week-old KP mice with adenoviruses expressing Cre (Adeno-Cre; FIG. 10A). At 10 weeks after infection, mice were treated with a single dose of 1 mg/kg bortezomib, a maximum tolerated dose that has been shown to inhibit the proteasome and NF-κB activity in mice (19, 35). Individual lung tumors were monitored using micro-computed tomography (microCT) imaging prior to treatment (D0) and 4 days post treatment (D4). As shown in FIGS. 3A and B, a single dose of bortezomib resulted in significant tumor regression (55.4% average decrease in tumor volume at D4 compared with D0), whereas vehicle control-treated mice showed a 47.2% average increase in tumor volume. Thus, established tumors with Kras mutations and loss of p53 function are acutely sensitive to treatment with bortezomib.

To determine whether bortezomib affects tumors from the K model, which retains functional p53, we initiated lung tumorigenesis in KrasLSL-G12D/wt mice with Adeno-Cre and treated the mice with a single 1 mg/kg dose of bortezomib at 20 weeks post infection (FIG. 10B). As shown in FIGS. 3C and D, K tumors did not regress upon treatment with bortezomib. Because tumors from KP mice had enhanced NF-κB p65 nuclear localization when compared with K tumors (30), the different therapeutic response between KP and K tumors suggests that the effect of bortezomib may depend on the tumor genotype or the basal NF-κB activity.

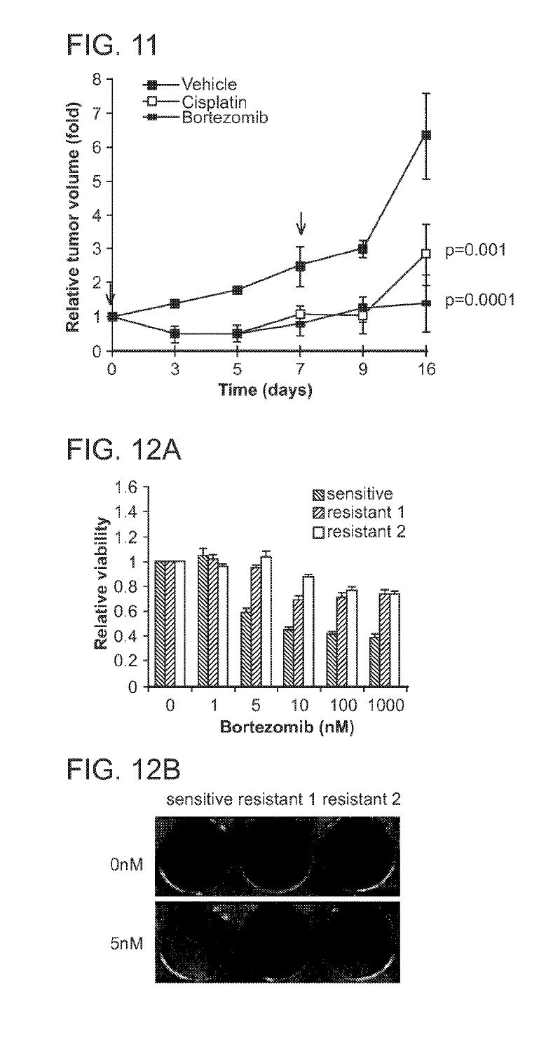

To compare the efficacy of bortezomib to that of other lung cancer therapies, we injected KP lung tumor cells s.c. into immunocompromised mice, allowed tumors to form, and then treated the mice with bortezomib. As shown in FIG. 11, bortezomib markedly reduced tumor volumes in the short term and diminished tumor progression to a similar extent as cisplatin, a first-line chemotherapy for lung cancer (2).

### Example 4

### Bortezomib Induces Apoptosis in KP Lung Tumors

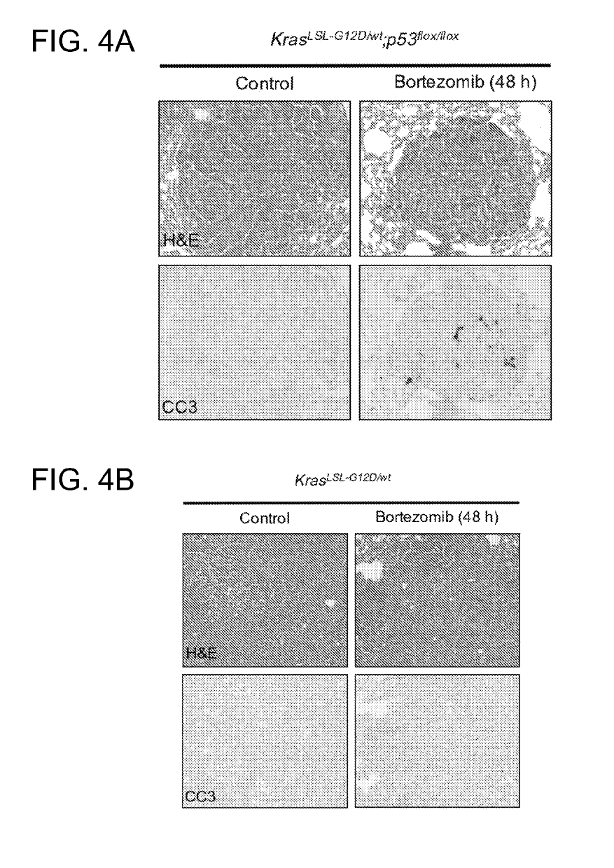

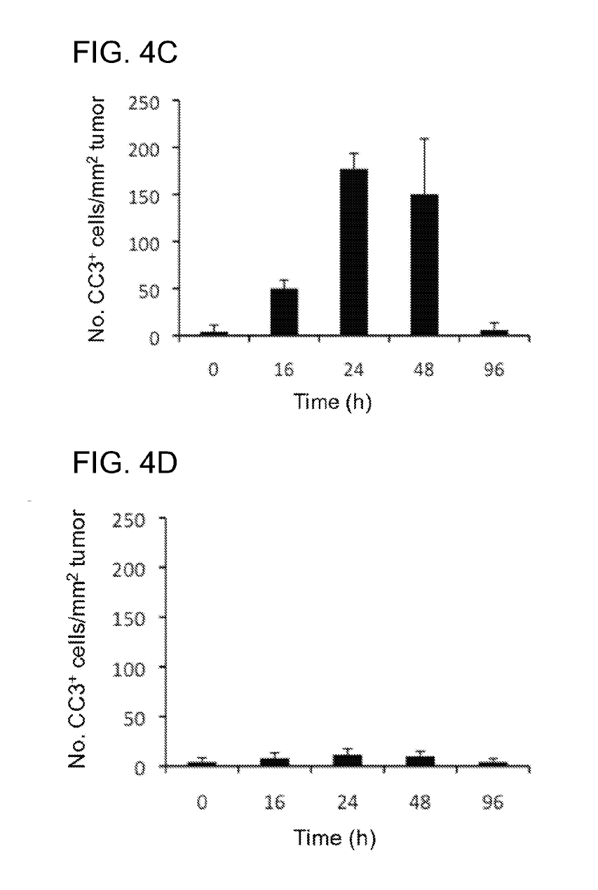

To address the mechanism of bortezomib-induced tumor regression, we stained control or bortezomib-treated KP and K tumor sections with an antibody that recognizes CC3. In KP tumors, we observed an increase in the number of apoptotic cells at 48 hours after a single dose of bortezomib (FIG. 4A). These results suggest that in the context of oncogenic Kras expression and p53 loss, bortezomib treatment leads to apoptosis both in vitro (FIG. 1C) and in vivo. To investigate the kinetics of bortezomib's effects in vivo, we stained tumor sections derived from a time-course experiment with antibodies to CC3. As shown in FIG. 4C, the CC3+ cell number peaked at 24 and 48 hours post treatment and diminished at 96 hours. This transient increase might be caused by the short half-life of bortezomib in vivo (36). Using a cohort of KrasLSL-G12D/wt mice, we asked whether bortezomib induces apoptosis in K-only tumors. In all the time points assayed, we did not detect a substantial number of apoptotic cells in bortezomib-treated tumors (FIGS. 4B and D). These results reinforced the importance of genetic context in the response to bortezomib and, in particular, the role of p53 mutation in conferring sensitivity to NF-κB inhibition.

### Example 5

### Bortezomib Increases Survival in KP Mice

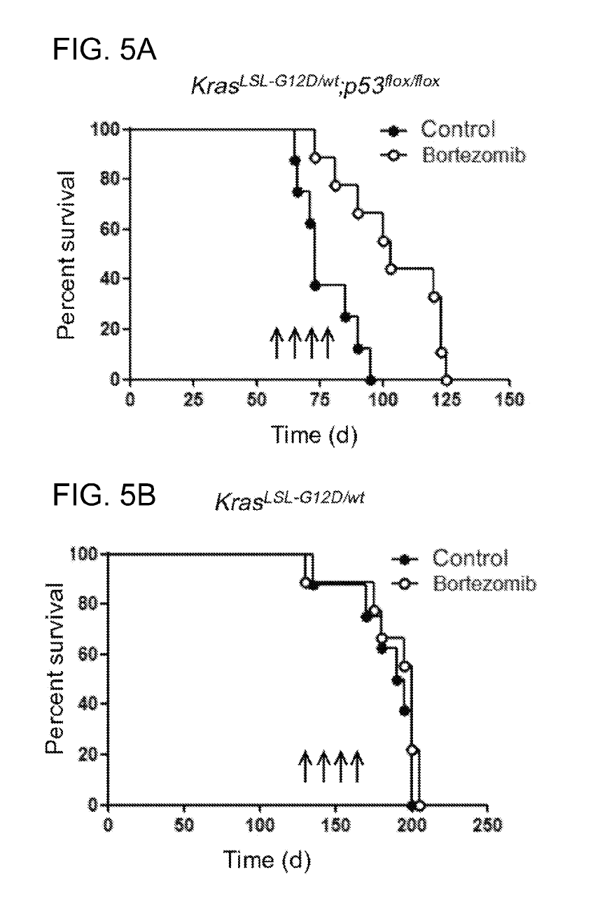

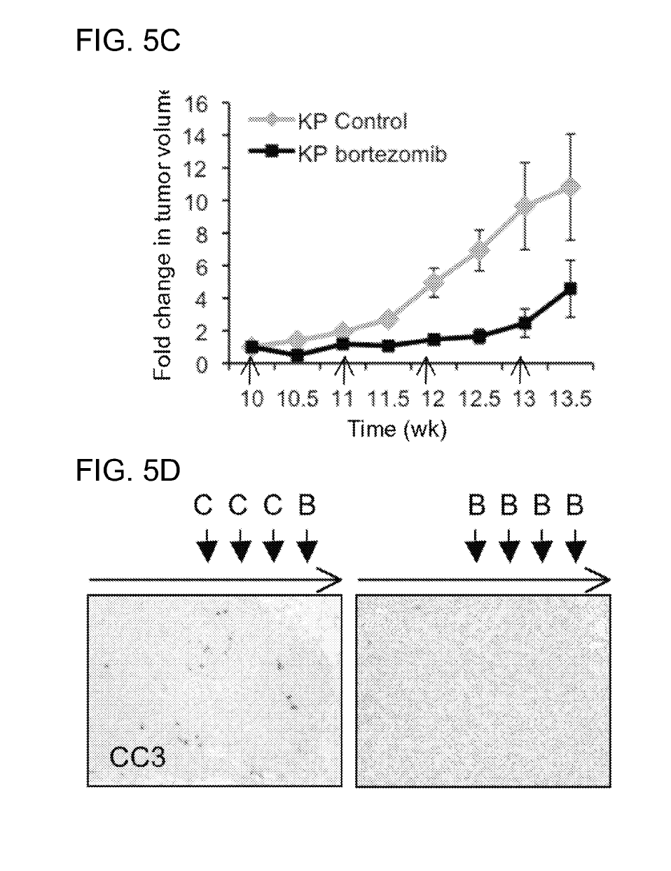

To analyze the long-term effects of bortezomib therapy, we investigated whether a 4-dose regimen of bortezomib (19) could prolong survival of tumor-bearing mice. Using a cohort of KP mice, we treated tumor-bearing animals 8 weeks after Adeno-Cre infection with bortezomib once a week for 4 weeks (FIG. 10A). As shown in FIG. 5A, KP mice treated with 4 doses of bortezomib survived significantly longer (104.2±19.8 days) than did control-treated mice (76.8±10.6 days; P=0.001). In contrast, consistent with the lack of tumor regression and cell death in KrasLSL-G12D/wt mice observed after short-term bortezomib treatment (FIGS. 3 and 4), no improvement in survival was observed in the KrasLSL-G12D/wt cohort (FIG. 5B; P=0.36). This finding suggests that loss of p53, although a predictor of poor prognosis in some cancer therapies, still permits therapeutic benefits from bortezomib and indicates that this agent and possibly other NF-κB inhibitors may work selectively in tumors with high basal NF-κB activity.

### Example 6

### Acquired Resistance Arises in KP Tumors after Bortezomib Treatment

Although the 4-dose regimen of bortezomib prolonged survival in the KP lung tumor model, the treated mice eventually succumbed to their disease (FIG. 5A). To examine whether bortezomib-treated tumors relapse after repeated therapy, we administered bortezomib to another cohort of tumor-bearing KP mice once a week for 4 weeks. Tumor response was measured by twice-weekly microCT to determine the volume of individual lung tumors. As we had observed with short-term microCT imaging (FIG. 3A), bortezomib treatment led to rapid KP tumor regression after the first dose (10.5 weeks; FIG. 5C) and delayed tumor growth, compared with vehicle control. However, by the 4th dose (13 weeks; FIG. 5C), tumors had become insensitive to treatment, suggesting that they had acquired resistance to the drug.

To investigate whether KP tumors present at the end of the treatment regimen were resistant to bortezomib-induced apoptosis, we treated KP mice, as described above, with 3 doses of bortezomib or vehicle control. After an additional week, mice were treated with a final dose of bortezomib and sacrificed 48 hours later. Tumors from mice that had received previous bortezomib treatments no longer demonstrated significant CC3 staining in response to a final dose of the drug (FIG. 5D, right), when compared with acutely treated naïve tumors (FIG. 5D, left). These data further suggest that the pretreated tumors had acquired resistance to bortezomib.

### Example 7

### An Orthotopic Lung Tumor Model for Imaging Response and Resistance to Bortezomib

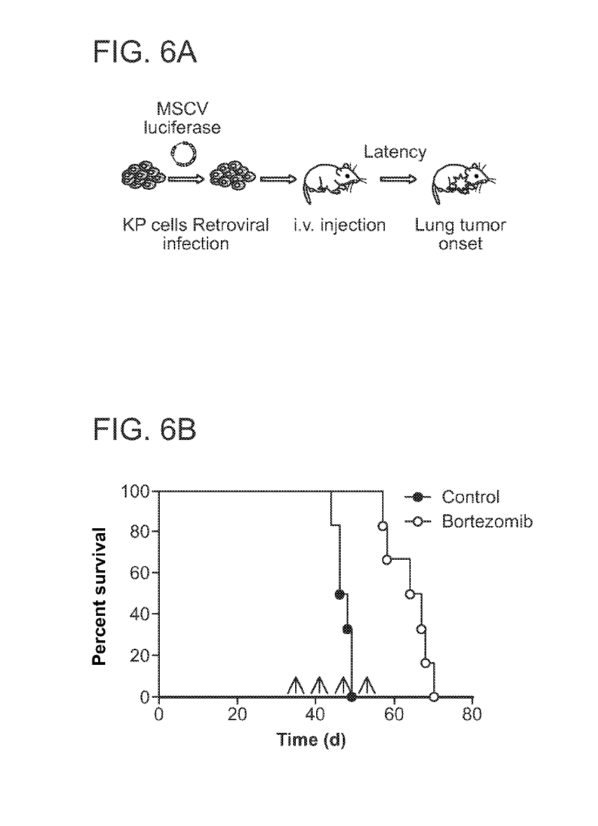

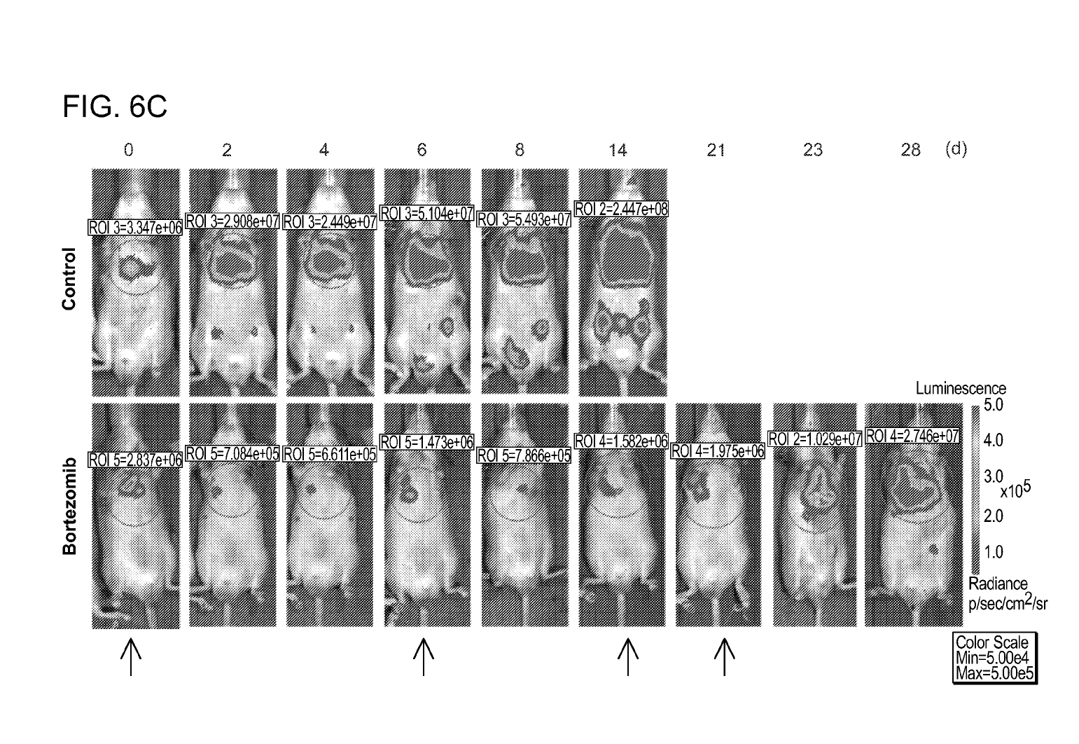

To facilitate imaging of the bortezomib treatment response, we adapted a transplantation-based orthotopic lung cancer mouse model (37). As outlined in FIG. 6A, we infected KP cell lines with a retrovirus expressing firefly luciferase. The cells were transplanted into immunocompetent recipient mice by i.v. injection, resulting in the development of in situ lung tumors that could be imaged and quantified by bioluminescence imaging.

To determine whether bortezomib therapy was effective in the orthotopic model, we transplanted 10,000 KP cells into host mice and treated the mice 5 weeks later, either with control or with a weekly 4-dose regimen of bortezomib (37). As in the autochthonous setting, in this model bortezomib treatment significantly increased survival, compared with control treatment (average survival: 47.0±2.0 days for control and 64.0±5.4 days for bortezomib; P=1.4×10⁻⁵; FIG. 6B). Bioluminescence imaging revealed a marked tumor regression at day 2 and day 4 after bortezomib treatment (FIG. 6C)—also reminiscent of the bortezomib-mediated lung tumor regression in the autochthonous model (FIG. 3A). After the 2nd and 3rd bortezomib treatments, the lung tumor signal still decreased or stabilized. However, as with the tumor relapse detected in the autochthonous model, a 4th dose of bortezomib was ineffective (FIG. 6C), suggesting the relapsed tumors had become refractory to drug treatment.

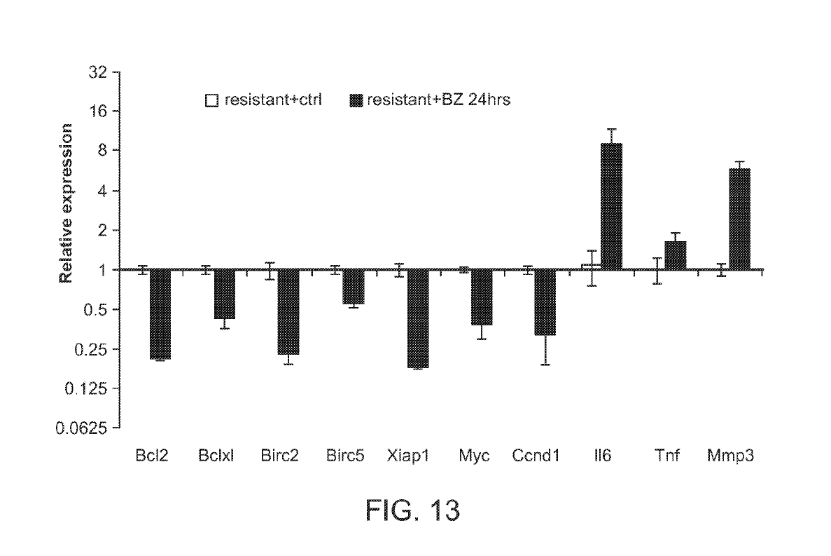

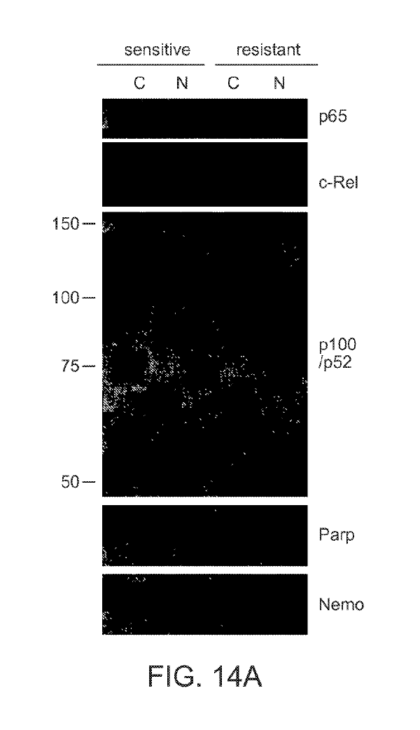

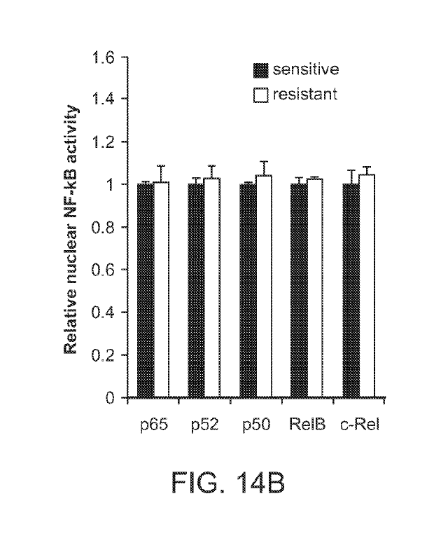

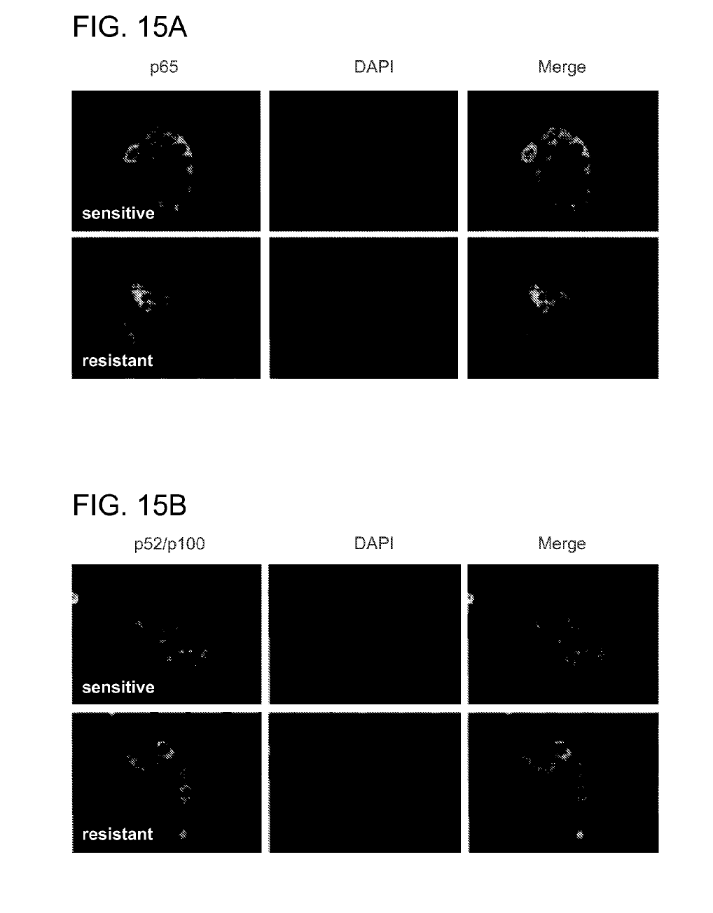

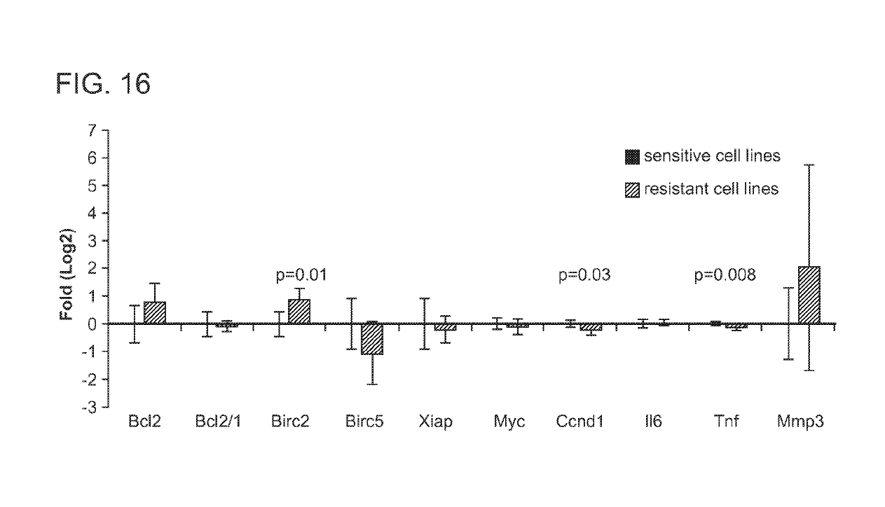

To test whether the onset of bortezomib resistance was accompanied by increased NF-κB activity, we generated cell lines from orthotopic KP tumors that were treated either with 4 doses of bortezomib (resistant cell lines) or with vehicle controls (sensitive cell lines). As shown in FIG. 12, resistant cells exhibited higher viability and increased colony formation, compared with sensitive cells, upon bortezomib treatment (FIGS. 12A and B). Of importance, when treated with bortezomib in culture, resistant cells showed robust downregulation of NF-κB target genes regulating apoptosis and survival (FIG. 13), indicating bortezomib was still effective in inhibiting the NF-κB pathway. Sensitive and resistant cell lines were next used to evaluate the activity of both the canonical and the noncanonical NF-κB pathways. Immunoblots, ELISA, and immunofluorescence analysis showed similar levels of cytoplasmic and nuclear NF-κB subunits in the resistant and sensitive cells (FIGS. 14 and 15). Moreover, transcriptional profiling of 10 NF-κB target genes did not reveal a globally increased expression of NF-κB targets in resistant tumor cells (FIG. 16). These data suggest that cells generated from bortezomib-resistant lung tumors harbor levels of basal NF-κB activity similar to those in sensitive cells.

### Example 8

### The NF-κB Inhibitor Bay-117082 Shows Therapeutic Efficacy In Vivo

To expand the scope of pharmacologic inhibition of NF-κB, we next tested a compound that was developed as an inhibitor of IKK. IKK-mediated IκB phosphorylation is required for IκB degradation and NF-κB activation (8), and Bay-117082 is a small-molecule compound that inhibits this IKK kinase activity (23). Like bortezomib, Bay-117082 has been shown to suppress NF-κB signaling in cells and mice (23, 24). Unlike bortezomib, which affects multiple cellular activities in addition to NF-κB, the use of Bay-117082 provides an extra degree of selectivity for inhibiting this pathway.

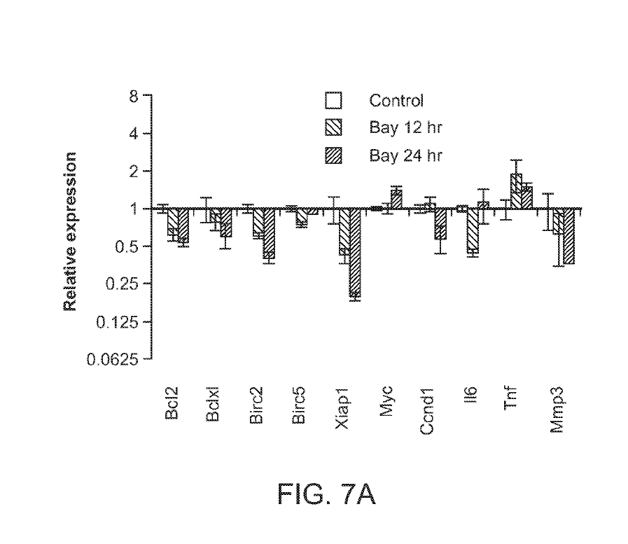

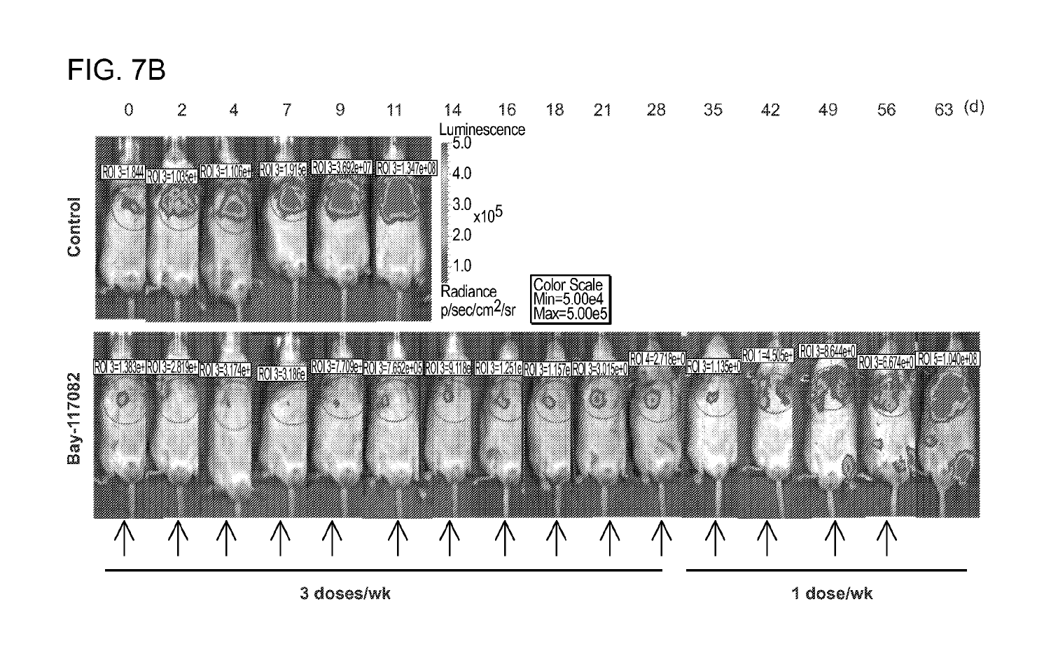

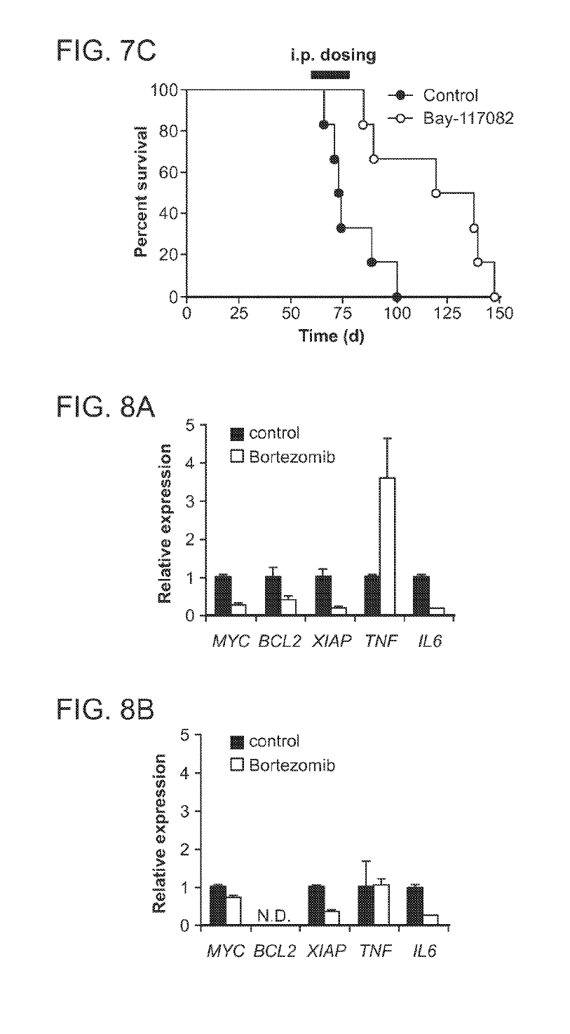

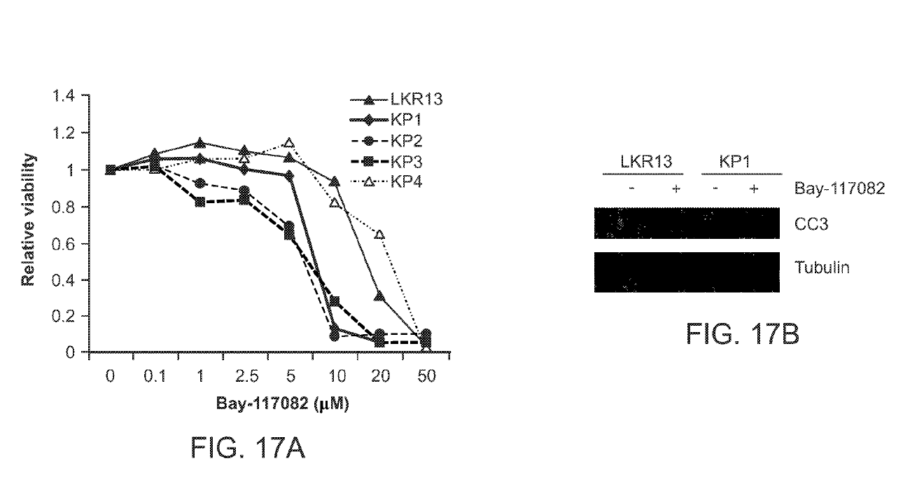

We found that Bay-117082 treatment induced caspase 3 cleavage and cell death in KP cell lines in vitro (FIG. 17). We assessed the expression of prosurvival NF-κB target genes in these cells after Bay-117082 treatment and found that Bay-117082 downregulates NF-κB target genes, such as Bcl2, Bclx1, and Xiap (FIG. 7A). Next we tested Bay-117082 in our orthotopic lung tumor model (FIG. 7B). Using bioluminescence imaging, we observed that Bay-117082 treatment significantly reduced lung tumor signal in the initial phase (FIG. 7B; 0-9 days) and delayed lung tumor progression until 42 days (FIG. 7B). However, treated tumors eventually became refractory to therapy and progressed in the lung and distant organs despite continuous Bay-117082 treatment (see 42-63 days in FIG. 7B). These data suggest that the relapsed tumors acquired resistance to the drug.

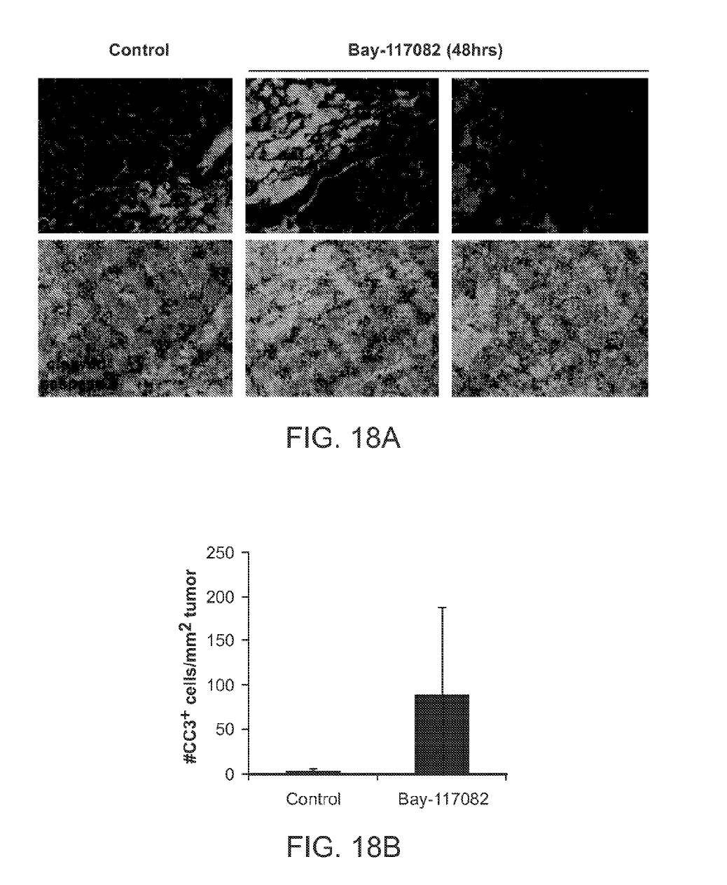

Finally, to gain insight into the survival benefit of Bay-117082, we treated a cohort of KP mice (8 weeks post Adeno-Cre) with Bay-117082 (10 mg/kg), 3 times per week by i.p. injection for 4 weeks, a dose previously shown to inhibit NF-κB in mice (24). As seen in FIG. 7C, the KP mice treated with Bay-117082 survived significantly longer than mice treated with vehicle control (79.0±13.2 days for control and 120.2±27.0 days for Bay-117082; P=0.008). In a short-term treatment, BAY-117082 led to apoptosis, as indicated by cleavage of caspase 3 (FIG. 18). Altogether, these results indicate that IKK inhibition has therapeutic efficacy in lung cancer.

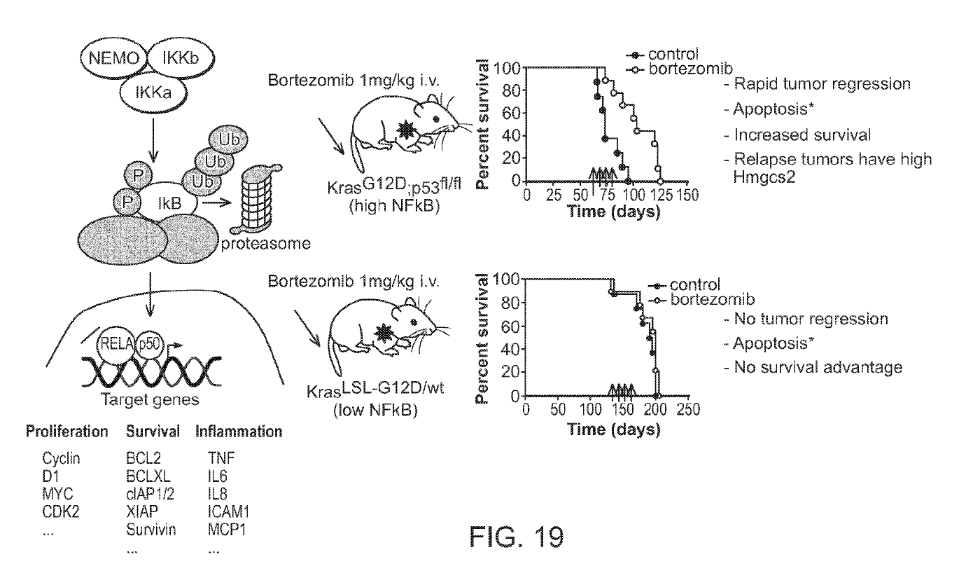

Mice having either high or low levels of NF-κB were treated with 1 kg/mg IV bortezomib and examined for tumor progression, apoptosis and survival. The mice are Kras^{G120};p53^{fl/fl} (high NF-κB) or KrasL^{St-G12Dwt} (low NF-κB). The results of the study are shown in FIG. 19. The top graph (Kras^{G120}; p53^{fl/fl}) showed rapid tumor progression, apoptosis, and increased survival. The relapsed tumors have high Hmcgs2. The graph on the bottom (KrasL^{St-G12Dwt}) demonstrates that mice with low NF-κB had no tumor regression, apoptosis and no survival advantage.

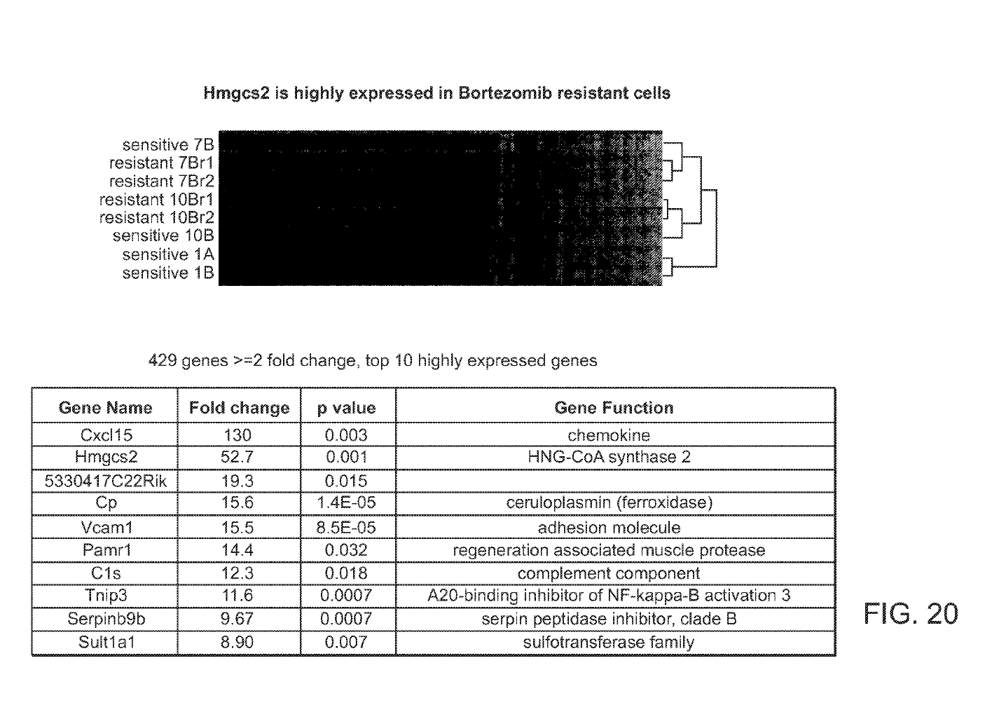

Levels of gene expression in bortezomib resistant cells was examined in order to identify mechanisms involved in NF-κB regulation of tumor cells. FIG. 20 is a blot and graph showing the results of expression in various genes in bortezomib resistant cells. The blot at the top of the page demonstrates that Hmgcs2 is highly expressed in these resistant cells. 429 genes demonstrated a greater than two fold change in expression. The top ten highly expressed genes in the bortezomib resistant cells are shown in the graph at the bottom of FIG. 20.

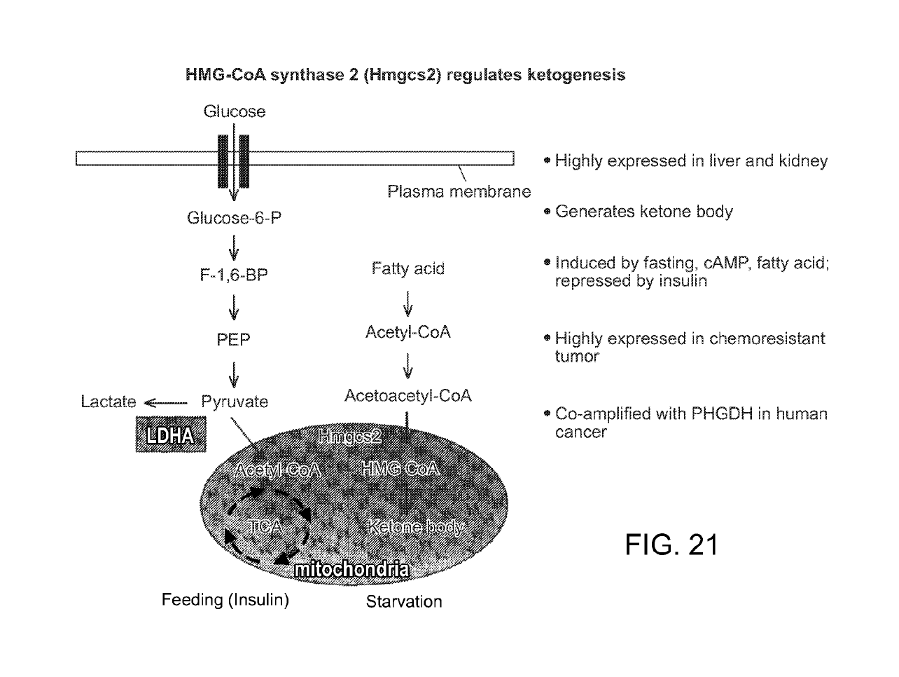

FIG. 21 is a schematic diagram demonstrating the mechanism through which Hmgcs2 regulates ketogenesis. The gene is highly expressed in kidney and liver and generates ketone bodies. It is induced by fasting, cAMP, fatty acid and repressed by insulin. It is highly expressed in chemoresistant tumors and is co-amplified with PHGDH in human cancer.

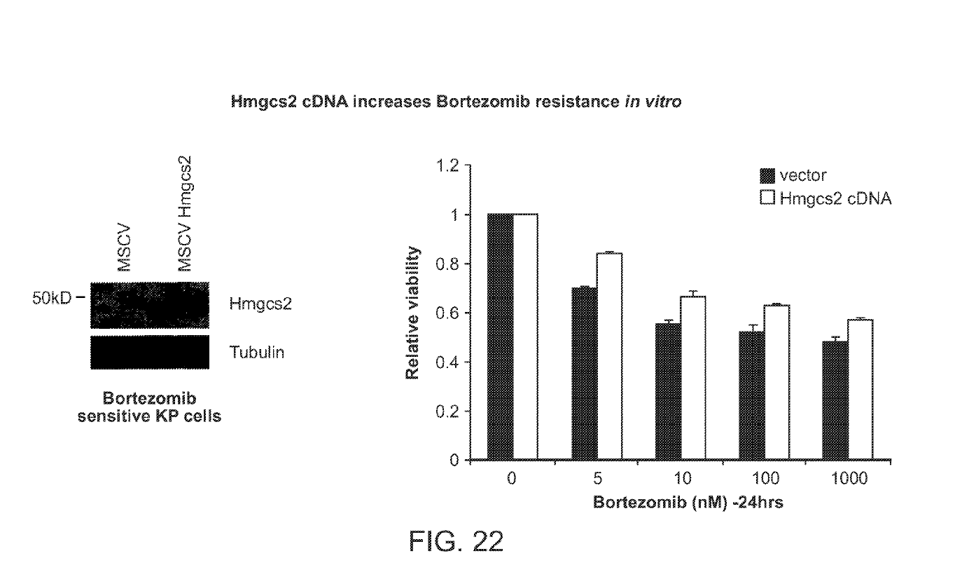

The expression levels of Hmgsc2 were examined more closely in bortezomib resistant cells. FIG. 22 shows a blot and a graph of the results of Hmgsc2 expression in bortezomib resistant cells in vitro. The data demonstrate that Hmgsc2 expression levels increases in these cells. The blot shown in the left hand panel and the graph is shown in the right hand panel.

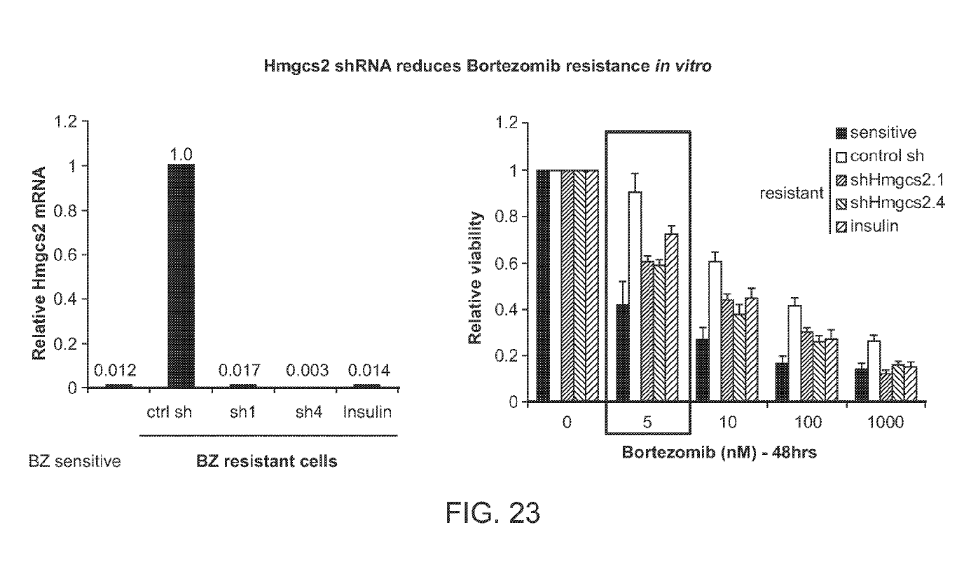

The effects of Hmgsc2 expression in bortezomib resistant cells was examined to determine if Hmgsc2 would be a good therapeutic target for the treatment of NF-kB regulated lung cancers. FIG. 23 is a set of graphs demonstrating that Hmgsc2 shRNA reduces bortezomib resistance in vitro. The graph on the left hand panel shows the relative amount of Hmgsc2 mRNA in bortezomib resistant cells when the cells are treated with Hmgsc2 shRNA, a control shRNA or insulin (positive control). As shown in the data the control levels of Hmgsc2 are quite high in the control and low in the other samples. The graph on the right hand side of the page shows the relative viability in bortezomib resistant cells at 48 hours treated under the conditions described above (in bortezomib resistant cells when the cells are treated with Hmgsc2 shRNA, a control shRNA or insulin). The shRNA to Hmgsc2 significantly reduced cell viability.

These studies evaluated the efficacy of pharmacologically targeting NF-κB in lung cancer. Our data showed that bortezomib, used as a single agent, provided significant survival advantage in a KRasG12D-driven p53-deficient lung cancer model. Bay-117082, an IKK inhibitory compound, also provided a survival advantage, although at a more frequent dosing schedule than that of the bortezomib regimen under the conditions tested. Our study provides evidence that the NF-κB pathway is a therapeutic target in lung cancer.

Our investigation highlights the value of mouse models in translating genetic knowledge into novel and improved cancer therapies. Our autochthonous model not only recapitulates important genetic and pathologic features of human lung adenocarcinoma but also provides a physiological tumor microenvironment to study therapeutic response. The orthotopic model, a variant of this approach, is based on the isolation of mouse lung adenocarcinoma cells from genetically tractable primary mouse lung tumors, followed by their seeding into the lungs of immunocompetent recipient mice (37). This latter model is also important for its ability to accelerate cancer treatment studies in mice, by allowing rapid imaging of therapeutic response and ex vivo genetic modification of cells by introduction of short hairpin RNAs or cDNAs. Using both systems, we have shown that mouse models, when combined with in vivo imaging and tumor biomarker analysis, can serve as a powerful platform to identify and validate novel cancer therapies. These mouse models will provide valuable preclinical information to be cross-compared with clinical trial data in human patients. Such studies could potentially dissect molecular mechanisms of selected drugs and identify biomarkers to predict patient response.

We have shown that bortezomib treatment induced apoptosis in lung tumors driven by activated Kras and lacking p53. Apoptosis may be one of the mechanisms underlying the significant decrease in lung tumor burden achieved by this drug in the KP model. We performed molecular characterization in cultured KP cells to show that bortezomib reduced expression of antiapoptotic NF-κB target genes (e.g., Bcl2, Bclx1, Birc2, and Xiap; FIG. 1B). This result is consistent with a prosurvival function of NF-κB in normal and cancer cells (8). Previous studies have developed inhibitors for Bcl2 family proteins (ABT-737; ref. 39) and cIAP1 (40, 41) as novel cancer therapies, but considering the simultaneous suppression of many antiapoptotic genes observed in bortezomib-treated cells (FIG. 1B), NF-κB inhibition appears to provide a promising approach to lower the apoptosis threshold in cancer cells. Because human tumors often upregulate NF-κB signaling to gain resistance to chemotherapy (12), NF-κB inhibitors may also serve as chemosensitizing agents in combination therapies.

Of note, bortezomib and Bay-117082 have differential effects in the transcriptional profile of certain NF-κB targets in vitro. For example, Bcl2 and Myc inhibition was more robust upon bortezomib treatment, whereas Xiap inhibition was stronger upon Bay-117082 treatment (FIG. 1B and FIG. 7A). Although Bay-117082-treated mice survived slightly longer than the bortezomib-treated cohort, these 2 groups were not statistically significant (P=0.103), and this effect may be due to a more frequent dosing schedule with Bay-117082 (3 injections per week) than with bortezomib. Our treatment data suggested that the efficacy of bortezomib is dependent on the genetic context of lung tumors. Our previous study showed that genetic inhibition of NF-κB by a IκB super-repressor (a dominant negative form of IκB) or knockdown of p65/RelA or NEMO preferentially triggered cell death in KP cells. but not in 3TZ or LKR13 cells (30). In this treatment study, KP cells also showed greater bortezomib sensitivity than did 3TZ or Kras-only cells. In vivo, KP tumors with high NF-κB activity were sensitive to bortezomib, whereas Kras-only tumors with lower activity were not responsive, which is consistent with clinical data showing that an NF-κB signature in multiple myeloma patients is associated with a better treatment outcome with bortezomib (22). Phase II clinical trials indicated that bortezomib has modest effects in advanced NSCLC patients previously treated with chemotherapy (42). Our observations that bortezomib sensitivity correlates with NF-κB activity suggest that NF-κB is a major target of this drug and NF-κB pathway activity may serve as a biomarker to predict the therapeutic response of bortezomib or other NF-κB inhibitory drugs.

In addition to inhibiting NF-κB, bortezomib and Bay-117082 have known multitargeted effects. Bortezomib can also stabilize the CDK inhibitors p21 and p27 (43), whereas Bay-117082 can stimulate the stress-activated protein kinases, p38 and JNK-1 (23). New classes of more selective NF-κB inhibitors, such as ATP analog IKK inhibitors, will improve efforts to drug the NF-κB pathway in cancer. Moreover, the therapeutic inhibition of NF-κB has thus far been viewed with caution owing to this pathway's diverse functions in different physiological contexts such as the immune system. Despite these cautions, bortezomib has been extensively used in the clinic with manageable side effects.

We further observed that prolonged bortezomib treatment led to resistance in KP lung tumors. Acquired bortezomib resistance has been reported in the literature (44), and our results are in agreement with clinical findings that multiple myelomas initially responsive to bortezomib often relapse and become resistant to the drug (44). Several studies have suggested possible mechanisms of bortezomib resistance, such as (1) mutations or overexpression of the PSMB5 subunit of 26S proteasome (45), (2) overexpression of HSP27 (46), and (3) increased activity of the aggresome pathway (44). Of interest, basal NF-κB activity is not increased in bortezomib-resistant lung tumor cell lines, at least in vitro (FIGS. 12-16). Our studies establish a physiologically relevant system to explore the mechanisms of bortezomib resistance in lung cancer.

In summary, we have characterized therapeutic response and resistance to NF-κB inhibitors in several mouse models of lung cancer. In vivo treatment with bortezomib or Bay-117082 significantly reduced tumor volume and increased survival in mice with lung tumors associated with high NF-κB activity. However, repeated treatment resulted in the emergence of drug-resistant tumors.

### REFERENCE LIST

1 Herbst, R S., Heymach, J. V. and Lippman, S. M. Lung cancer, N. EngL J. Med., 359: 13671380, 2008.
2 Oliver, T. G., Mercer, K. L., Sayles, L. C., Burke, J. R, Mendus, D., LovejoY, K. S., Cheng, M. H., Subramanian, A., Mu, D., Powers, S., Crowley, D., Bronson, R T., Whittaker, C. A., Bhutkar, A., Lippard, S. l., Golub, T., Thomale, J., Jacks, T. and Sweet-Cordero, E. A. Chronic cisplatin treatment promotes enhanced damage repair and tumor progression in a mouse model of lung cancer, Genes Dev., 24: 837-852, 2010.
3 Downward, J. Targeting RAS signalling pathways in cancer therapy, Nat. Rev. Cancer, 3: 11-22, 2003.
4 Sordella, R., Bell, D. W., Haber, D. A. and Settleman, J. Gefitinib-sensitizing EGFR mutations in lung cancer activate anti-apoptotic pathways, Science, 305: 1163-1167, 2004.
5 Gerber, D. E. and Minna, J. D. ALK inhibition for non-small cell lung cancer: from discovery to therapy in record time, Cancer Cell, 18: 548-551, 2010.
6 Baud, Y. and Karin, M. Is NF-kappaB a good target for cancer therapy? Hopes and pitfalls, Nat. Rev. Drug Discov., 8: 33-40, 2009.
7 Karin, M., Yamamoto, Y. and Wang, Q. M. The IKK NF-kappa B system: a treasure trove for drug development, Nat. Rev. Drug Discov., 3: 17-26, 2004.
8 Hayden, M. S. and Ghosh, S. Signaling to NF-kappaB, Genes Dev., /8: 2195-2224, 2004.
9 Baltimore, D. Discovering NF-kappaB, Cold Spring Harb. Perspect. Biol., 1: a000026, 2009.
10 Basseres, D. S. and Baldwin, A. S. Nuclear factor-kappaB and inhibitor of kappaB kinase pathways in oncogenic initiation and progression, Oncogene, 25: 6817-6830, 2006.
11 Hanahan, D. and Weinberg, R A. The hallmarks of cancer, Cell, 100: 57-70, 2000.
12 Nakanishi, C. and Toi, M. Nuclear factor-kappaB inhibitors as sensitizers to anticancer drugs, Nat. Rev. Cancer, 5: 297-309, 2005.
13 Annunziata, C. M., Davis, R E., Demchenko, Y., Bellamy, W., Gabrea, A., Zhan, F., Lenz, G., Hanamura, l., Wright, G., Xiao, W., Dave, S., Hurt, E. M., Tan, B., Zhao, H., Stephens, O., Santra, M., Williams, D. R., Dang, L., Barlogie, B., Shaughnessy, J. D., Jr., Kuehl, W. M. and Staudt, L. M. Frequent engagement of the classical and alternative NF-kappaB pathways by diverse genetic abnormalities in multiple myeloma, Cancer Cell, 12: 115-130, 2007.
14 Kan, Z., Jaiswal, B. S., Stinson, J., Janakiraman, V., Bhatt, D., Stern, H. M., Yue, P., Haverty, P. M., Bourgon, R, Zheng, J., Moorhead, M., Chaudhuri, S., Tomsho, L. P., Peters, B. A., Pujara, K., Cordes, S., Davis, D. P., Carlton, V. E., Yuan, W., Li, L., Wang, W., Eigenbrot, C., Kaminker, J. S., Eberhard, D. A., Waring, P., Schuster, S. C., Modrusan, Z., Zhang, Z., Stokoe, D., de Sauvage, F. J., Faham, M. and Seshagiri, S. Diverse somatic mutation patterns and pathway alterations in human cancers, Nature, 466: 869-873, 2010.
15 Compagno, M., Lim, W. K., Grunn, A., Nandula, S. V., Brahmachary, M., Shen, Q., Bertoni, F., Ponzoni, M., Scandurra, M., Califano, A., Bhagat, G., Chadbum, A., la-Favera, R and Pasqualucci, L. Mutations of multiple genes cause deregulation of NF-kappaB in diffuse large B-celllymphoma, Nature, 459: 717-721, 2009.
16 Lam, L. T., Davis, R E., Pierce, J., Hepperle, M., XU, Y., Hottelet, M., Nong, Y., Wen, D., Adams, J., Dang, L. and Staudt, L. M. Small molecule inhibitors of lkappaB kinase are selectively toxic for subgroups of diffuse large B-celllymphoma defined by gene expression profiling, Clin. Cancer Res., 11: 28-40, 2005.
17 Gilmore, T. D. and Herscovitch, M. Inhibitors of NF-kappaB signaling: 785 and counting, Oncogene, 25: 6887-6899, 2006.
18 Chauhan, D., Hideshima, T. and Anderson, K. C. Proteasome inhibition in multiple myeloma: therapeutic implication, Annu. Rev. Pharmacol. Toxicol., 45: 465-476, 2005.
19 Adams, J., Palombella, V. J., Sausville, E. A., Johnson, J., Destree, A., Lazarus, D. D., Maas, J., Pien, C. S., Prakash, S. and Elliott, P. J. Proteasome inhibitors: a novel class of potent and effective antitumor agents, Cancer Res., 59: 2615-2622, 1999.
20 LeBlanc, R., Catley, L. P., Hideshima, T., Lentzsch, S., Mitsiades, C. S., Mitsiades, N., Neuberg, D., Goloubeva, O., Pien, C. S., Adams, J., Gupta, D., Richardson, P. G., Munshi, N. C. and Anderson, K. C. Proteasome inhibitor PS-341 inhibits human myeloma cell growth in vivo and prolongs survival in a murine model, Cancer Res., 62: 4996-5000, 2002.
21 Richardson, P. G., Mitsiades, C., Hideshima, T. and Anderson, K. C. Bortezomib: proteasome inhibition as an effective anticancer therapy, Annu. Rev. Med., 57: 33-47, 2006.
22 Mulligan, G., Mitsiades, C., Bryant, B., Zhan, F., Chng, W, J., Roels, S., Koenig, E., Fergus, A., Huang, Y., Richardson, P., Trepicchio, W. L., Broyl, A., Sonneveld, P., Shaughnessy, J. D., Jr., Bergsagel, P. L., Schenkein, D., Esseltine, D. L., Boral, A. and Anderson, K. C. Gene expression profiling and correlation with outcome in clinical trials of the proteasome inhibitor bortezomib, Blood, 109: 3177-3188, 2007.
23 Pierce, J. W., Schoenleber, R., Jesmok, G., Best, J., Moore, S. A., Collins, T. and Gerritsen, M. E. Novel inhibitors of cytokine-induced IkappaB alpha phosphorylation and endothelial cell adhesion molecule expression show anti-inflammatory effects in vivo, J. Biol. Chem., 272: 21096-21103, 1997.
24 Keller, S. A., Hernandez-Hopkins, D., Vider, J., Ponomarev, V., Hyjek, E., Schattner, E. J. and Cesarman, E. NF-kappaB is essential for the progression of KSHV- and EBV-infected lymphomas in vivo, Blood, 107: 3295-3302, 2006.
25 Schmitt, C. A., Fridman, J. S., Yang, M., Lee, S., Baranov, E., Hoffman, R. M. and Lowe, S. W. A senescence program controlled by p53 and p 161NK4a contributes to the outcome of cancer therapy, Cell, 109: 335-346, 2002.
26 Engelman, J. A., Chen, L., Tan, X., Crosby, K., Guimaraes, A. R., Vpadhyay, R., Maira, M., McNamara, K., Perera, S. A., Song, Y., Chirieac, L. R., Kaur, R., Lightbown, A., Simendinger, J., Li, T., Padera, R. F., Garcia-Echeverria, C., Weissleder, R., Mahmood, V., Cantley, L. C. and Wong, K. K. Effective use of PI3K and MEK inhibitors to treat mutant Kras GI2D and PIK3CAHI047Rmurine lung cancers, Nat. Med., 14: 1351-1356, 2008.
27 Jackson, E. L., Willis, N., Mercer, K., Bronson, R. T., CrowleY, D., Montoya, R., Jacks, T. and Tuveson, D. A. Analysis of lung tumor initiation and progression using conditional expression of oncogenic K-ras, Genes Dev., 15: 3243-3248, 2001.
28 DuPage, M., Dooley, A. L. and Jacks, T. Conditional mouse lung cancer models using adenoviral or lentiviral delivery of Cre recombinase, Nat. Protoc., 4: 1064-1072, 2009.
29 Tuveson, D. A., Shaw, A. T., Willis, N. A., Silver, D. P., Jackson, E. L., Chang, S., Mercer, K. L., Grochow, R., Hock, H., CrowleY, D., Hingorani, S. R., Zaks, T., King, C., Jacobetz, M. A., Wang, L., Bronson, R. T., Orkin, S. H., DePinho, R. A. and Jacks, T. Endogenous oncogenic Kras(G 12D) stimulates proliferation and widespread neoplastic and developmental defects, Cancer Cell, 5: 375-387, 2004.
30 Meylan, E., Dooley, A. L., Feldser, D. M., Shen, L., Turk, E., Ouyang, C. and Jacks, T. Requirement for NF-kappaB signalling in a mouse model of lung adenocarcinoma, Nature, 462: 104-107, 2009.
31 Basseres, D. S., Ebbs, A., Levantini, E. and Baldwin, A. S. Requirement of the NF-kappaB subunit p65/RelA for K-Ras-induced lung tumorigenesis, Cancer Res., 70: 3537-3546, 2010.
32 Deng, J., Fujimoto, J., Ye, X. F., Men, T. Y., Van Pelt, C. S., Chen, Y. L., Lin, X. F., Kadara, H., Tao, Q., Lotan, D. and Lotan, R. Knockout of the tumor suppressor gene Gprc5a in mice leads to NF-kappaB activation in airway epithelium and promotes lung inflammation and tumorigenesis, Cancer Prev. Res. (Phila), 3: 424-437, 2010.
33. Hideshima T, Richardson P, Chauhan D, Palombella V J, Elliott P J, Adams J, et al. The proteasome inhibitor PS-341 inhibits growth, induces apoptosis, and overcomes drug resistance in human multiple myeloma cells. Cancer Res 2001; 61:3071-6.
34. Jackson E L, Olive K P, Tuveson D A, Bronson R, Crowley D, Brown M, et al. The differential effects of mutant p53 alleles on advanced murine lung cancer. Cancer Res 2005; 65:10280-8.
35. Sunwoo J B, Chen Z, Dong G, Yeh N, Crowl B C, Sausville E, et al. Novel proteasome inhibitor PS-341 inhibits activation of nuclear factor-kappa B, cell survival, tumor growth, and angiogenesis in squamous cell carcinoma. Clin Cancer Res 2001; 7:1419-28.
36. Dubey S, Schiller J H. Three emerging new drugs for NSCLC: pemetrexed, bortezomib, and cetuximab. Oncologist 2005; 10:282-91.
37. Doles J, Oliver T G, Cameron E R, Hsu G, Jacks T, Walker G C, et al. Suppression of Rev3, the catalytic subunit of Pol{zeta}, sensitizes drug-resistant lung tumors to chemotherapy. Proc Natl Acad Sci USA 2010; 107:20786-91.
38. Barbie D A, Tamayo P, Boehm J S, Kim S Y, Moody S E, Dunn I F, et al. Systematic RNA interference reveals that oncogenic KRAS-driven cancers require TBK1. Nature 2009; 462:108-12.
39. Oltersdorf T, Elmore S W, Shoemaker A R, Armstrong R C, Augeri D J, Belli B A, et al. An inhibitor of Bcl-2 family proteins induces regression of solid tumours. Nature 2005; 435:677-81.
40. Varfolomeev E, Blankenship J W, Wayson S M, Fedorova A V, Kayagaki N, Garg P, et al. IAP antagonists induce autoubiquitination of c-IAPs, NF-kappaB activation, and TNFalpha-dependent apoptosis. Cell 2007; 131:669-81.
41. Vince J E, Wong W W, Khan N, Feltham R, Chau D, Ahmed A U, et al. IAP antagonists target cIAP1 to induce TNFalpha-dependent apoptosis. Cell 2007; 131:682-93.
42. Fanucchi M P, Fossella F V, Belt R, Natale R, Fidias P, Carbone D P, et al. Randomized phase II study of bortezomib alone and bortezomib in combination with docetaxel in previously treated advanced non-small-cell lung cancer. J Clin Oncol 2006; 24:5025-33.
43. Mack P C, Davies A M, Lara P N, Gumerlock P H, Gandara D R. Integration of the proteasome inhibitor PS-341 (Velcade) into the therapeutic approach to lung cancer. Lung Cancer 2003; 41 Suppl 1:S89-96.
44. Kumar S, Rajkumar S V. Many facets of bortezomib resistance/susceptibility. Blood 2008; 112:2177-8.
45. Oerlemans R, Franke N E, Assaraf Y G, Cloos J, van Zantwijk I, Berkers C R, et al. Molecular basis of bortezomib resistance: proteasome subunit beta5 (PSMB5) gene mutation and overexpression of PSMB5 protein. Blood 2008; 112:2489-99.
46. Chauhan D, Li G, Shringarpure R, Podar K, Ohtake Y, Hideshima T, et al. Blockade of Hsp27 overcomes Bortezomib/proteasome inhibitor PS-341 resistance in lymphoma cells. Cancer Res 2003; 63:6174-7.
47. Kirsch D G, Grimm J, Guimaraes A R, Wojtkiewicz G R, Perez B A, Santiago P M, et al. Imaging primary lung cancers in mice to study radiation biology. Int J Radiat Oncol Biol Phys 2010; 76:973-7.
48. Xue W, Krasnitz A, Lucito R, Sordella R, Vanaelst L, Cordon-Cardo C, et al. DLC1 is a chromosome 8p tumor suppressor whose loss promotes hepatocellular carcinoma. Genes Dev 2008; 22:1439-44.
49. Zender L, Xue W, Zuber J, Semighini C P, Krasnitz A, Ma B, et al. An oncogenomics-based in vivo RNAi screen identifies tumor suppressors in liver cancer. Cell 2008; 135:852-64.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method, comprising
determining a level of NF-κB in a subject having lung cancer, and administering to the subject an effective amount of an NF-κB inhibitor to treat the subject if the subject has a higher than normal level of NF-κB.

2. The method of claim 1, wherein the NF-κB inhibitor is bortezomib.

3. The method of claim 1, wherein the NF-κB inhibitor is Bay-117082.

4. The method of claim 1, further comprising detecting a level of a marker gene expressed in a sample of cancerous tissue from the subject.

5. The method of claim 4, wherein the marker gene is selected from the group consisting of HMG-CoA synthase 2, Cxcl15, 5330417C22Rik, Cp, Vcam1, Pamr1, C1s, Tnip3, Serpinb98, and Sult1a1.

6. The method of claim 5, wherein when the level of the marker gene is at least twice a baseline level, the subject is administered a chemotherapeutic agent.

7. The method of claim 1, wherein the subject has lung cancer with mutations in Kras and p53.

8. A method, comprising detecting a level of a marker gene expressed in a sample of cancerous tissue from a subject having lung cancer, wherein if the level of the marker gene is at least twice a baseline level the cancerous tissue is resistant to treatment with a NF-κB inhibitor, wherein the marker gene is selected from the group consisting of genes listed in Table 1, and developing a therapeutic strategy for the subject based on the level of the marker gene.

9. The method of claim 8, wherein the marker gene is selected from the group consisting of HMG-CoA synthase 2, Cxcl15, 5330417C22Rik, Cp, Vcam1, Pamr1, C1s, Tnip3, Serpinb98, and Sult1a1.

10. The method of claim 8, wherein the subject has lung cancer with mutations in Kras and p53.

11. The method of claim 8, wherein the marker gene is HMG-CoA synthase 2.

12. The method of claim 8, further comprising administering the subject a chemotherapeutic agent.

13. A method, comprising
administering to a subject having lung cancer an NF-κB inhibitor and an HMG-CoA synthase 2 inhibitor in an effective amount to treat the cancer.

14. The method of claim 13, wherein the HMG-CoA synthase 2 inhibitor is an siRNA to HMG-CoA synthase 2.

15. The method of claim 13, wherein the HMG-CoA synthase 2 inhibitor is an shRNA to HMG-CoA synthase 2.

16. The method of claim 13, wherein the subject has lung cancer with mutations in Kras and p53.

17. A method, comprising
administering to a subject having lung cancer an NF-κB inhibitor and an NF-κB responsive agent in an effective amount to treat the cancer.

18. The method of claim 17, wherein the an NF-κB responsive agent is an siRNA.
